SEQUENCE LISTING (9 sequences)

SEQ ID NO: 1 - DNA, 17 bp, Artificial Sequence
  Synthetic oligonucleotide
cttttgcaggtcatcgg

SEQ ID NO: 2 - DNA, 31 bp, Artificial Sequence
  Synthetic Oligonucleotide
acagaattcgccccggcctggtacactgcca

SEQ ID NO: 3 - DNA, 23 bp, Artificial Sequence
  Synthetic oligonucleotide
tccaaggagctgcaggcggcgca

SEQ ID NO: 4 - DNA, 23 bp, Artificial Sequence
  Synthetic oligonucleotide
tccaaggagctgcaggcggcgca

SEQ ID NO: 5 - DNA, 18 bp, Artificial Sequence
  Synthetic oligonucleotide
acttttgcaggtcatcgg

SEQ ID NO: 6 - DNA, 25 bp, Artificial Sequence
  Synthetic oligonucleotide
ccccggcctggtacactgccaggct

SEQ ID NO: 7 - DNA, 56 bp, Artificial Sequence
  Synthetic oligonucleotide
cgcgatgccgatgacctgcagaagtgcctggcagtgtaccaggccggggcccgcga

SEQ ID NO: 8 - DNA, 18 bp, Artificial Sequence
  Synthetic oligonucleotide
gcttttgcaggtcatcgg

SEQ ID NO: 9 - DNA, 56 bp, Artificial Sequence
  Synthetic oligonucleotide
cgcgatgccgatgacctgcagaagcgcctggcagtgtaccaggccggggcccgcga

(19) United States
(12) Patent Application Publication
(10) Pub. No.: US 2005/0003355 A1
(43) Pub. Date: Jan. 6, 2005

(54) Single nucleotide polymorphism analysis using surface invasive cleavage reactions

(76) Inventors: Manchun Lu, Jersey City, NJ (US); Jeff G. Hall, Madison, WI (US); Michael R. Shortreed, Portage, WI (US); Liman Wang, Lansdale, PA (US); Travis Berggren, Madison, WI (US); Priscilla Wilkins Stevens, Evanston, IL (US); David M. Kelso, Wilmette, IL (US); Victor Lyamichev, Madison, WI (US); Bruce Neri, Madison, WI (US); Lloyd M. Smith, Madison, WI (US)

Correspondence Address: DEWITT ROSS & STEVENS S.C., 8000 EXCELSIOR DR, SUITE 401, MADISON, WI, 53717-1914, US

(21) Appl. No.: 10/420,247

(22) Filed: Apr. 22, 2003

Related U.S. Application Data
(60) Provisional application No. 60/374,546, filed on Apr. 22, 2002.

(51) Int. Cl. C12Q 1/68

(52) U.S. Cl. 435/6

(57) ABSTRACT

Disclosed are a method and a corresponding composition of matter for detecting single nucleotide polymorphisms. The composition of matter includes a metal substrate, a probe oligonucleotide immobilized on the substrate; and an upstream oligonucleotide either immobilized on the substrate or in a solution in contact with the substrate. The probe oligonucleotide and the upstream oligonucleotide participate cooperatively in an invasive cleavage reaction when the substrate is contacted with a cleavage agent and a target nucleic acid. Under proper reactions conditions, the probe oligonucleotide, the upstream oligonucleotide, the target nucleotide, and the cleavage agent cooperative result in the formation of a cleavage product at or near the location of a single nucleotide polymorphism of interest.

### CROSS-REFERENCE TO RELATED APPLICATIONS

Priority is hereby claimed to provisional application Ser. No. 60/374,546, filed Apr. 22, 2002, and incorporated herein by reference.

### FEDERAL FUNDING

This work was supported by Grant R01HG02298 from National Institutes of Health.

### REFERENCES AND INCORPORATION BY REFERENCE

Complete bibliographic citations for the references cited herein are contained in a section titled “REFERENCES,” immediately preceding the claims. All of the documents listed in the “REFERENCES” section are incorporated herein.

### BACKGROUND

Single nucleotide polymorphisms (SNPs) are the most abundant and stable type of variations found in the human genome. SNPs appear in the human genome with an estimated frequency of one polymorphic nucleotide per kilobase.¹ The abundance of SNPs enables them to be used as genetic markers in linkage and association studies aimed at identifying and characterizing genes involved in biological function and human disease.²⁻⁵ However, estimates also suggest that for such studies to be successful in analyzing common disease genes, it may be necessary to characterize several hundred thousand SNPs, in perhaps hundreds or even thousands of individuals.⁶ This limitation on the use of SNPs has presented a tremendous challenge and obstacle to performing such linkage studies.

Recent work describes an invasive cleavage reaction for SNP scoring. This approach has a number of desirable features, including the ability to analyze genomic DNA directly, with high accuracy, robustness, and in an isothermal homogeneous format.^{7,8} The reaction is based upon cleavage of a unique secondary structure formed between two adjacent oligonucleotides, one referred to as the “upstream” oligonucleotide and the other as the “probe” oligonucleotide, both oligonucleotides being hybridized to a target DNA sequence. The nucleotide at the 3′ end of the upstream oligonucleotide is designed to overlap at least one base into the downstream duplex formed by the probe and the target strand. The unpaired region on the 5′ end of the probe, or “flap,” along with an immediate downstream paired nucleotide can then be removed by a class of structure-specific 5′-exonucleases.⁹ Absolute complementarity between the probe and the target sequence at the position of overlap is required for efficient enzymatic cleavage, which provides a cleavage rate at least 300 times higher than for a non-complementary substitute.¹⁰

This huge difference in cleavage rate is the basis for the discrimination of single base differences in the target DNA strand. The use of a thermostable 5′-exonuclease allows the reaction to be performed near the melting temperature (Tm) of the hybridization region between the probe and target strand. Thus, when the reaction is performed completely in solution phase, with an excess amount of probe oligonucleotide present, a cleaved probe will quickly be replaced by an uncleaved one. The probe oligonucleotides exchange on and off the target strand for a reaction run near the Tm, which results in a linear accumulation of cleavage product with respect to both time and target strand concentration. Under optimal operating conditions, approximately 3,000 cleaved probes can be generated per target molecule in about 90 minutes.⁷

Unlike the target-amplification employed in most current SNP scoring technologies, the signal-amplification format of this assay eliminates carryover contamination which can occur in PCR.¹¹⁻¹⁴ The combination of sequence-specific probe hybridization and structure-specific enzymatic cleavage imparts a high degree of specificity to the reaction, sufficient for the robust detection of a single nucleotide change directly from nanogram amounts of genomic DNA in a serial two-step invasive cleavage reaction.⁸ This assay is in routine use today for clinical SNP screening.^{15, 16}

### SUMMARY OF THE INVENTION

The invasive cleavage reaction described hereinabove has been adapted to a variety of different formats, including the use of mass spectrometric¹⁷ and microparticle-based¹⁸ detection of the cleavage products. One convenient way to monitor the reaction in a homogenous format is by using a Fluorescence Resonance Energy Transfer (FRET) mechanism. In this approach, which forms part of the subject invention, the energy emitted by a donor fluorophore is transferred to a nearby acceptor dye, and dissipates as heat, rather than being emitted as fluorescence.¹⁹ During the reaction, cleavage physically separates the donor fluorophore from the acceptor dye on the probe, eliminating the dye-quenching and generating a fluorescence signal.

A powerful approach to analyzing SNPs, an approach that is the subject of the present invention, is to implement the invasive cleavage reaction described above in an immobilized, surface array format. By preparing immobilized DNA arrays on surfaces where each element of the array contains a particular SNP-specific probe, adding a single sample of human genomic DNA to the surface would lead to the formation of the invasive cleavage structure at every site on the surface corresponding to an SNP allele in the genome being analyzed (See FIG. 1). The invasive cleavage reaction gives rise to an increase in fluorescence at that element of the array, indicating the presence of the corresponding SNP allele in the target DNA. In essence, the use of the planar surface format parallelizes the invasive cleavage reaction. As a consequence, each different SNP allele in the genome is queried, in massively parallel fashion, by the corresponding sites on the surface. A DNA array containing, for example, one million immobilized probe sites would thus permit 500,000 bi-allelic SNPs to be analyzed in a single step.

The upstream oligonucleotide, which is also required for the invasive cleavage reaction, either could be added in solution, or alternatively could be co-immobilized on the surface along with the probe (see FIG. 2C for a schematic).¹⁸ Co-immobilizing the upstream oligonucleotide also obviates the issues associated with having many different upstream oligonucleotides interacting in solution in a multiplexed format.

The Examples presented herein demonstrate the operability of performing invasive cleavage reactions on planar substrates. As shown in the Examples, the reaction can be accomplished using either synthetic oligonucleotides or a PCR amplicon as a target nucleic acid. A polymorphism in codon 158 of the human ApoE gene, which plays a key role in the transport and metabolism of plasma cholesterol and triglycerides,²⁰ was used as a model system. The surface cleavage reaction was studied by measuring the surface fluorescence intensity as a function of probe cleavage. A theoretical model was then developed that relates these two parameters (fluorescence intensity vs. probe cleavage). Variables affecting the rate of the surface invasive cleavage reaction were also examined.

### BRIEF DESCRIPTION OF THE FIGURES

FIG. 1: Two sets of oligonucleotides are required for characterizing codon 158 of the human ApoE gene using the invasive cleavage reaction of the present invention. The bases at the polymorphic site are thymidine (T) and cytosine (C). In either reaction set, the 3′ terminal nucleotide of the upstream oligonucleotide overlaps (or invades) the first base pair of the downstream probe-target duplex (A-T for the T-allele target and G-C for the C-allele target). Note that the probe oligonucleotides (“T-allele probe” and “C-allele probe”) are immobilized on a gold surface. The 5′ exonuclease specifically cleaves the probes at the positions marked by the arrows. Cleavage separates the dabcyl-fluorescein FRET pair (denoted “D” and “F,” respectively) and disables the quenching action of the dabcyl. In the first invasive cleavage reaction strategy, only the probe oligonucleotide is attached to the surface. In the second invasive cleavage reaction strategy, both the 3′ end of the probe oligonucleotide and the 5′ end of the upstream oligonucleotide are immobilized. The merits of each reaction strategy are detailed in the text. Oligonucleotides attached to the surface are previously modified with spacer phosphoramidites and free thiols. The free thiols of the oligonucleotide react with maleimide groups on modified gold surfaces to immobilize the oligonucleotides on the surface.

FIGS. 2A, 2B, & 2C: Fluorescence images and schematics of both surface invasive cleavage reaction strategies. FIG. 2A-Control: The probe oligonucleotide 10 is attached to the surface and the upstream oligonucleotide 12 is added in solution. No target was added to this reaction and thus, no significant signal increase was observed. FIG. 2B-Strategy 1 Reaction: The probe oligonucleotide 10 is attached to the surface and the upstream oligonucleotide 12 is added in solution. Upon addition of 50 pM target 14 (T-allele) and incubation at 54.5° C. for 24 hours, the fluorescence intensity increased, by an average factor of 3.5, due to formation of cleavage product 10′. FIG. 2C-Strategy 2 Reaction: Both the probe oligonucleotide 10 and the upstream oligonucleotide 12 are attached to the surface. Upon addition of 50 pM target 14 (T-allele) and incubation at 54.5° C. for 24 hours, the fluorescence intensity increased, on average, by a factor of 2.3, due to formation of cleavage product 10′. The control experiment for the co-immobilized surface generated similar results to the control experiment described in FIG. 2A.

FIG. 3: This histogram details the fluorescence intensity changes of the surface invasive cleavage reactions shown in FIGS. 2B and 2C. In strategy 1, only the probe oligonucleotide is attached to the surface; whereas in strategy 2, both the probe and the upstream oligonucleotides are attached to the surface. Four spots (˜150 pixels total) were statistically analyzed for each strategy. The error bars represent the standard deviation of those pixels.

FIG. 4: Synthetic target and PCR amplicons of codon 158 of the human ApoE gene were genotyped using the first surface invasive cleavage reaction strategy (probe immobilized, upstream oligonucleotide in solution). Three experiments were performed with 1 pmol total of synthetic target in combinations representing the three possible SNP genotypes. One experiment was performed using approximately 0.5 pmole of a single-stranded PCR amplicon. Post-reaction fluorescence images of each surface are shown. The percentage signal change for each sample is shown in the corresponding histogram. Relative (rather than absolute) fluorescence intensity is used because of some minor variability in the amount of probe at each spot of the array. Two spots (˜75 pixels total) for each sample were statistically analyzed for each time point. The error bars represent the standard deviation of those pixels.

FIG. 5: The average surface fluorescence intensity changes as a function of probe cleavage fraction. A series of samples were prepared to simulate different stages in the progress of the surface invasive cleavage reaction. The data points shown are the average of the quadruplet of each sample, and the error bars represent the standard deviation of the measured intensities from the ˜80 pixels in each quadruplet. The straight line connecting the 0% and 100% points is a model based on the assumption of intramolecular only energy transfer with constant FRET efficiency. The curved dashed line is a model which takes into account both intramolecular and intermolecular FRET. It is this second model that appears to describe the data quite accurately.

### DEFINITIONS

The terms “cleavage agent” and “cleavage means” are synonymous and designate any agent or combination of agent(s) capable of cleaving a cleavage structure, including but not limited to enzymes. Generally, a cleavage agent is an enzyme or chemical agent having nuclease activity, such as those enzymes described in U.S. Pat. Nos. 6,090,606; 5,795,763; and 5,614,402. Suitable cleavage agents for use in the present invention are available commercially from Third Wave Technologies, Inc. (Madison, Wis.). Cleavage agents includes native DNA polymerases having 5′ nuclease activity (e.g., Taq DNA polymerase, E. coli DNA polymerase I) and, more specifically, modified DNA polymerases having 5′ nuclease but lacking DNA synthetic activity. The ability of 5′ nucleases to cleave naturally-occurring structures in nucleic acid templates (structure-specific cleavage) is useful to detect internal sequence differences in nucleic acids without prior knowledge of the specific sequence of the nucleic acid. In this manner, they are structure-specific enzymes. “Structure-specific nucleases” or “structure-specific enzymes” are enzymes which recognize specific secondary structures in a nucleic molecule and cleave these structures. A cleavage agent cleaves a nucleic acid molecule in response to the formation of cleavage structures; it is not necessary that the cleavage agent cleaves the cleavage structure at any particular location within the cleavage structure. Cleavage agents are not restricted to enzymes having solely 5′ nuclease activity. The cleavage agent may include nuclease activity provided from a variety of sources including, “CLEAVASE”-brand enzymes, FEN-1 endonucleases (including RAD2 and XPG proteins), Taq DNA polymerase and E. coli DNA polymerase I.

The term “cleavage products” as used herein, refers to products generated by the reaction of a cleavage agent with a cleavage structure (i.e., the treatment of a cleavage structure with a cleavage agent).

The term “cleavage structure” as used herein, refers to a structure which is formed by the interaction of a probe oligonucleotide and a target nucleic acid to form a duplex, the resulting structure being cleavable by a cleavage agent. The cleavage structure is a substrate for specific cleavage by the cleavage agent. (This is in contrast to a nucleic acid molecule which is a substrate for non-specific cleavage by agents such as phosphodiesterases, which cleave nucleic acid molecules without regard to secondary structure and in the absence of a duplexed structure.)

The terms “complementary” or “complementarity” are used in reference to polynucleotides (i.e., a sequence of nucleotides such as an oligonucleotide or a target nucleic acid) related by the canonical base-pairing rules (T/A; G/T). For example, the sequence “A-G-T” is totally complementary to the sequence “T-C-A.” Complementarity may be “partial,” in which only some of the bases are matched according to the canonical pairing rules. “Total” or “complete” complementarity indicates that the canonical pairing rules are followed exactly.

An oligonucleotide is present in “excess” relative to another oligonucleotide (or target nucleic acid sequence) if the first oligonucleotide is present at a higher molar concentration that the other oligonucleotide (or target nucleic acid sequence). When an oligonucleotide, such as a probe oligonucleotide, is present in a cleavage reaction in excess relative to the concentration of the complementary target nucleic acid sequence, the reaction may be used to indicate the amount of the target nucleic acid present. Typically, when present in excess, the probe oligonucleotide will be present in at least a 100-fold molar excess; e.g., typically at least 1 pmole of each probe oligonucleotide would be used when the target nucleic acid sequence was present at about 10 fmoles or less.

The term “homology” refers to a degree of identity between two polynucleotides. There may be partial homology or complete homology. A partially identical sequence is a sequence that is less than 100% identical to another sequence.

The term “hybridization” denotes the pairing of complementary nucleic acids. Hybridization and the strength of hybridization (i.e., the strength of the association between the nucleic acids) is impacted by such factors as the degree of complementarity between the nucleic acids, the stringency of the conditions involved, the Tₘ of the hybrid, and the G to C ratio within the nucleic acids.

“Hybridization methods” involve the annealing of a complementary sequence to the target nucleic acid (i.e., the sequence to be detected; the detection of this sequence may be by either direct or indirect means). The ability of two polymers of nucleic acid containing complementary sequences to find each other and anneal through base pairing interaction is a well-recognized phenomenon. The initial observations of the “hybridization” process by Marmur & Lane, Proc. Natl. Acad. Sci. USA 46:453 (1960) and Doty et al., Proc. Natl. Acad. Sci. USA 46:461 (1960) have been followed by the refinement of this process into an essential tool of modern biology.

The term “invader oligonucleotide” or simply “invader” refers to an oligonucleotide which contains sequences at its 3′ end which are substantially the same as sequences located at the 5′ end of a probe oligonucleotide; these regions will compete for hybridization to the same segment along a complementary target nucleic acid. This term is synonymous with “upstream oligonucleotide.”

The term “label” as used herein refers to any atom or molecule which can be used to provide a detectable (and preferably quantifiable) signal, and which can be attached to a nucleic acid or protein. Labels may provide signals detectable by fluorescence, radioactivity, colorimetry, gravimetry, X-ray diffraction or absorption, magnetism, enzymatic activity, and the like. A label may be a charged moiety (positive or negative charge) or alternatively, may be neutral.

The term “non-target cleavage product” refers to a product of a cleavage reaction which is not derived from the target nucleic acid. As noted herein, in the methods of the present invention, cleavage of the cleavage structure occurs within the probe oligonucleotide. The fragments of the probe oligonucleotide generated by this target nucleic acid-dependent cleavage are “non-target cleavage products.”

“Nucleic acid sequence” as used herein refers to a nucleotide, oligonucleotide, and/or polynucleotide, and fragments or portions thereof, and to DNA or RNA of genomic or synthetic origin which may be single- or double-stranded, and represent the sense or antisense strand. Similarly, “amino acid sequence” as used herein refers to peptide or protein sequence.

The term “oligonucleotide” designates a molecule comprised of two or more deoxyribonucleotides or ribonucleotides, preferably at least 5 nucleotides, more preferably at least about 10-15 nucleotides and more preferably at least about 15 to 30 nucleotides. The exact size will depend on many factors, which in turn depends on the ultimate function or use of the oligonucleotide. The oligonucleotide may be generated in any manner, including chemical synthesis, DNA replication, reverse transcription, or a combination thereof.

The term “polymerization means” refers to any agent capable of facilitating the addition of nucleoside triphosphates to an oligonucleotide. Preferred polymerization means comprise DNA polymerases.

The term “polymorphic locus” or “polymorphism” designates a locus present in a population which shows variation between members of the population (i.e., the most common allele has a frequency of less than 0.95). In contrast, a “monomorphic locus” is a genetic locus having little or no variations seen between members of the population (generally taken to be a locus at which the most common allele exceeds a frequency of 0.95 in the gene pool of the population).

“Primer” refers to an oligonucleotide which is capable of acting as a point of initiation of synthesis when placed under conditions in which primer extension is initiated. A primer sequence need not reflect the exact sequence of the template. For example, a non-complementary nucleotide fragment may be attached to the 5′ end of the primer, with the remainder of the primer sequence being substantially complementary to the strand. Non-complementary bases or longer sequences can be interspersed into the primer, provided that the primer sequence has sufficient complementarity with the sequence of the template to hybridize and thereby form a template primer complex for synthesis of the extension product of the primer.

“Probe oligonucleotide” refers to an oligonucleotide which interacts with a target nucleic acid to form a cleavage structure in the presence or absence of an invader oligonucleotide. When annealed to the target nucleic acid, the probe oligonucleotide and target form a cleavage structure and cleavage occurs within the probe oligonucleotide. In the presence of an invader oligonucleotide upstream of the probe oligonucleotide along the target nucleic acid will shift the site of cleavage within the probe oligonucleotide (relative to the site of cleavage in the absence of the invader).

The term “reactant” is used herein in its broadest sense. The reactant can comprise an enzymatic reactant, a chemical reactant or ultraviolet light (ultraviolet light, particularly short wavelength ultraviolet light is known to break oligonucleotide chains). Any agent capable of reacting with an oligonucleotide to either shorten (i.e., cleave) or elongate the oligonucleotide is encompassed within the term “reactant.”

The term “sample” is used in its broadest sense. On one hand, it includes a specimen or culture (e.g., microbiological cultures). On the other hand, it includes both biological and environmental samples. Biological samples may be animal, including human, fluid, solid (e.g., stool) or tissue, as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagamorphs, rodents, etc. Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

A “single nucleotide polymorphism” is a “polymorphic locus” which displays a variation in the identity of a single nucleotide between members of the population.

The term “source of target nucleic acid” refers to any sample which contains nucleic acids (RNA or DNA). Particularly preferred sources of target nucleic acids are biological samples including, but not limited to blood, saliva, cerebral spinal fluid, pleural fluid, milk, lymph, sputum and semen.

“Stringency” is used in reference to the conditions of temperature, ionic strength, and the presence of other compounds, under which nucleic acid hybridizations are conducted. At “high stringency” conditions, nucleic acid base pairing will occur only between nucleic acid fragments that have a high frequency of complementary base sequences. At “weak” or “low” stringency, nucleic acids that are not completely complementary to one another will hybridize to one another.

The term “target nucleic acid” refers to a nucleic acid molecule which contains a sequence which has at least partial complementarity with at least a probe oligonucleotide and may also have at least partial complementarity with an invader oligonucleotide. The target nucleic acid may comprise single- or double-stranded DNA or RNA.

The term “thermostable” when used in reference to an enzyme, such as a 5′ nuclease, indicates that the enzyme is functional or active (i.e., can perform catalysis) at an elevated temperature, i.e., at about 55° C. or higher.

The term “Tₘ” denotes “melting temperature.” The melting temperature is the temperature at which, on average, one-half a population of double-stranded nucleic acid molecules becomes dissociated into pairs of complementary, single-stranded molecules. Several equations for estimating the Tₘ of nucleic acids is well known in the art. As indicated by standard references, a simple rule of thumb to estimate Tₘ is the equation: Tₘ=81.5+0.41 (% G+C), when a nucleic acid is in aqueous solution at 1 M NaCl. Other methods include more sophisticated computations that take structural characteristics, as well as sequence, into account.

The term “upstream oligonucleotide” is synonymous with “invader oligonucleotide,” defined hereinabove.

When two different, non-overlapping oligonucleotides anneal to different regions of the same linear complementary nucleic acid sequence, and the 3′ end of one oligonucleotide points towards the 5′ end of the other, the former is called the “upstream” oligonucleotide and the latter is called the “downstream” oligonucleotide.

The term “virus” refers to obligate, ultramicroscopic, intracellular parasites incapable of autonomous replication (i.e., replication requires the use of the host cell's machinery).

### DETAILED DESCRIPTION OF THE INVENTION

The structure-specific invasive cleavage reaction is a useful means for sensitive and specific detection of single nucleotide polymorphisms, or SNPs, directly from genomic DNA, without the need for prior target amplification. A new approach integrating this invasive cleavage assay and surface DNA array technology is disclosed herein. The inventive method can be used for large-scale SNP scoring in a parallel format. To demonstrate the invention, two surface invasive cleavage reaction strategies were designed and implemented for a model SNP system in codon 158 of the human ApoE gene. The upstream oligonucleotide, which is required for the invasive cleavage reaction, is either co-immobilized on the surface along with the probe oligonucleotide, or alternatively added in solution. The ability of this approach to discriminate a single base difference unambiguously was demonstrated using PCR-amplified human genomic DNA. A theoretical model relating the surface fluorescence intensity to the progress of the invasive cleavage reaction was developed, and agreed well with experimental results.

Thus, in a first embodiment, the invention is directed to a method of detecting SNPs. The method includes the steps of first providing the following items; a source of target nucleic acid molecules, at least one molecule of which comprises a first region and a second region, the second region being downstream of the first region, and further comprising a polymorphic nucleotide disposed between the first region and the second region, and wherein the first region, the polymorphic nucleotide, and the second region are contiguous; a probe oligonucleotide comprising a 5′-terminal nucleotide and a 3′-terminus, wherein the probe oligonucleotide is immobilized at or near its 3′ terminus to an inert substrate, and wherein the probe oligonucleotide is complementary to the first portion and the polymorphic nucleotide of the target nucleic acid, with the 5′-terminal nucleotide of the probe oligonucleotide corresponding to and complementary to the polymorphic nucleotide of the target nucleic acid; an upstream oligonucleotide comprising a 3′ terminal nucleotide and a contiguous 5′ portion, wherein the 5′ portion is complementary to the second portion of the target nucleic, and the 3′ terminal nucleotide corresponds to the polymorphic nucleotide in the target nucleic acid, and is or is not complementary thereto; and a cleavage agent.

With the provision of these items, the cleavage agent, the target nucleic acid, and the upstream oligonucleotide are then contacted to the immobilized probe oligonucleotide to create a reaction mixture under reaction conditions such that the probe oligonucleotide is annealed to the first region and the polymorphic nucleotide of the target nucleic acid and wherein at least the fraction of the 5′-portion of the upstream oligonucleotide is annealed to the second region of the target nucleic acid at a point contiguous to the polymorphic nucleotide in the target nucleic acid so as to create a cleavage structure. This causes cleavage of the cleavage structure, thereby generating non-target cleavage products immobilized on the inert support. Cleavage of the cleavage structure is then detected, whereby the polymorphic nucleotide in the target nucleic acid is detected.

Detecting cleavage of the cleavage structure can be done by any means known in the art or developed in the future for detecting the cleavage of nucleic acid molecules. Thus, cleavage can be detected using a means for detection selected from the group consisting of means for detecting fluorescence, means for detecting mass; means for detecting fluorescence energy resonance transfer, means for detecting radioactivity, means for detecting luminescence, means for detecting phosphorescence, means for detecting fluorescence polarization, and means for detecting charge.

For a complete description of invasive cleavage reactions conducted entirely in solution and the detection thereof, see U.S. Pat. Nos. 6,348,314; 6,090,543; 6,001,567; 5,994,069; 5,985,557; and 5,846,717, all of which are incorporated herein.

In the preferred method, the cleavage agent comprises a structure-specific nuclease, most preferably a thermostable structure-specific nuclease.

The target nucleic acid may comprise any type of nucleic acid, without limitation, including DNA, RNA, and modified forms thereof (e.g., nucleic acids containing modified bases, labels, binding moieties, spacers, linkers, heteroduplexes, etc.).

The source of the target nucleic acid is not important or critical to the functionality of the invention. The source from which the target nucleic acid originates can be selected, for example (and not by way of limitation) from the group comprising blood, saliva, cerebral spinal fluid, pleural fluid, milk, lymph, sputum, and semen.

The nature of the inert substrate also is not critical to the operation of the invention, so long as the support is inert to the various reagents used in the method. The preferred inert substrate is a thin metal layer, preferably a transition or noble metal, and most preferably gold. A non-limiting list of preferred metal substrates includes gold, silver, platinum, palladium, copper, nickel, and titanium.

The chemical means by which the oligonucleotides are secured to the surface also is not critical to the function of the invention, so long as the method chosen reliably immobilizes the nucleic acid to the substrate and leaves the immobilized nucleic acid capable of hybridizing with complementary nucleic acids contacted with the immobilized nucleic acid. For the preferred methodology, see the Examples and reference nos. 23 and 24.

A preferred route to immobilizing oligonucleotides on an inert substrate proceeds as follows: First, a self-assembled monolayer of a C₆ to C₆₀ alkanethiol, such as 11-mercaptoundecanoic acid (MUA), is formed on a gold-coated substrate, followed by electrostatic adsorption of a poly-L-lysine (PL) monolayer. This electrostatic reaction is caused by the attraction between the carboxylic acid groups of the MUA and the amine groups of the PL. Free amine groups on PL not involved in the electrostatic interaction with the acid-terminated surface are then reacted with a heterobifunctional linker, such as sulfosuccinimidyl 4-(N-maleimidomethyl) cyclohexane-1-carboxylate (SSMCC). This creates a thiol-reactive, maleimide-terminated surface that can covalently interact with thiol-modified DNA strands. The thiol-modified DNA strands are then covalently bonded to the substrate via the maleiumide-terminated surface.

Other permutations of the above-described SNP detection method are also included within the scope of the invention. Thus, the invention also includes, a method of detecting a single nucleotide polymorphism in a population of target nucleic acid molecules, wherein the method comprises: (a) providing: (i) a cleavage agent; (ii) a source of target nucleic acid molecules, at least one molecule of which comprises a first region and a second region, the second region being downstream of the first region, and further comprising a polymorphic nucleotide disposed between the first region and the second region, and wherein the first region, the polymorphic nucleotide, and the second region are contiguous; (iii) a probe oligonucleotide comprising a 5′-terminal nucleotide and a 3′-terminus, wherein the probe oligonucleotide is immobilized at or near its 3′ terminus to an inert substrate, and wherein the probe oligonucleotide is complementary to the first portion and the polymorphic nucleotide of the target nucleic acid, with the 5′-terminal nucleotide of the probe oligonucleotide corresponding to and complementary to the polymorphic nucleotide of the target nucleic acid; (iv) an upstream oligonucleotide comprising a 3′ terminal nucleotide, a contiguous 5′ portion, and a 5′ terminus, wherein the upstream oligonucleotide is immobilized at or near its 5′ terminus to the inert substrate at a point adjacent to the immobilized probe oligonucleotide, and wherein a fraction of the 5′ portion is complementary to the second portion of the target nucleic, and the 3′ terminal nucleotide corresponds to the polymorphic nucleotide in the target nucleic acid, and is or is not complementary thereto; and then (b) contacting the cleavage agent and the target nucleic acid to the immobilized probe oligonucleotide and the immobilized upstream oligonucleotide to create a reaction mixture under reaction conditions such that the probe oligonucleotide is annealed to the first region and the polymorphic nucleotide of the target nucleic acid and wherein at least the 5′ portion of the upstream oligonucleotide is annealed to the second region of the target nucleic acid at a point contiguous to the polymorphic nucleotide in the target nucleic acid so as to create a cleavage structure, and wherein cleavage of the cleavage structure occurs to generate non-target cleavage products immobilized on the inert support; and (c) detecting cleavage of the cleavage structure, whereby the polymorphic nucleotide in the target nucleic acid is detected.

The invention also includes a method comprising: (a) providing: (i) a cleavage agent; (ii) a source of target nucleic acid molecules, at least one molecule of which comprises a first region and a second region, the second region being downstream of the first region, and further comprising a polymorphic nucleotide disposed between the first region and the second region, and wherein the first region, the polymorphic nucleotide, and the second region are contiguous; (iii) a probe oligonucleotide comprising a 5′-terminal nucleotide and a 3′-terminal nucleotide, wherein the probe oligonucleotide is immobilized at or near one of the 5′- or 3′-terminal nucleotides to an inert substrate, and wherein the probe oligonucleotide is complementary to the first portion and the polymorphic nucleotide of the target nucleic acid, with the terminal nucleotide of the probe oligonucleotide not bound to the substrate corresponding to and complementary to the polymorphic nucleotide of the target nucleic acid; (iv) an upstream oligonucleotide comprising a 3′ terminal nucleotide, a 5′ terminal nucleotide, and 3′ portion and a 5′ portion; wherein one of the 3′- or 5′ portions is complementary to the second portion of the target nucleic, and one of the 3′- or 5′-terminal nucleotides corresponds to the polymorphic nucleotide in the target nucleic acid, and is or is not complementary thereto; and then (b) contacting the cleavage agent, the target nucleic acid, and the upstream oligonucleotide to the immobilized probe oligonucleotide to create a reaction mixture under reaction conditions such that the probe oligonucleotide is annealed to the first region and the polymorphic nucleotide of the target nucleic acid and wherein at least the fraction of the 3′ or 5′ portion of the upstream oligonucleotide is annealed to the second region of the target nucleic acid at a point contiguous to the polymorphic nucleotide in the target nucleic acid so as to create a cleavage structure, and wherein cleavage of the cleavage structure occurs to generate non-target cleavage products immobilized on the inert support; and (c) detecting cleavage of the cleavage structure, whereby the polymorphic nucleotide in the target nucleic acid is detected.

The invention further includes a method comprising: (a) providing: (i) a cleavage agent; (ii) a source of target nucleic acid molecules, at least one molecule of which comprises a first region and a second region, the second region being downstream of the first region, and further comprising a polymorphic nucleotide disposed between the first region and the second region, and wherein the first region, the polymorphic nucleotide, and the second region are contiguous; (iii) a probe oligonucleotide comprising a 5′-terminal nucleotide and a 3′-terminal nucleotide, wherein the probe oligonucleotide is immobilized at or near one of the 5′- or 3′-terminal nucleotides to an inert substrate, and wherein the probe oligonucleotide is complementary to the first portion and the polymorphic nucleotide of the target nucleic acid, with the terminal nucleotide of the probe oligonucleotide not bound to the substrate corresponding to and complementary to the polymorphic nucleotide of the target nucleic acid; (iv) an upstream oligonucleotide comprising a 3′ terminal nucleotide, a 5′ terminal nucleotide, and 3′ portion and a 5′ portion, wherein the upstream oligonucleotide is immobilized at or near one of the 5′- or 3′-terminal nucleotides to the inert substrate; wherein one of the 3′- or 5′ portions is complementary to the second portion of the target nucleic, and one of the 3′- or 5′-terminal nucleotides corresponds to the polymorphic nucleotide in the target nucleic acid, and is or is not complementary thereto; and then (b) contacting the cleavage agent and the target nucleic acid to the immobilized upstream oligonucleotide and the immobilized probe oligonucleotide to create a reaction mixture under reaction conditions such that the probe oligonucleotide is annealed to the first region and the polymorphic nucleotide of the target nucleic acid and wherein at least the fraction of the 3′ or 5′ portion of the upstream oligonucleotide is annealed to the second region of the target nucleic acid at a point contiguous to the polymorphic nucleotide in the target nucleic acid so as to create a cleavage structure, and wherein cleavage of the cleavage structure occurs to generate non-target cleavage products immobilized on the inert support; and (c) detecting cleavage of the cleavage structure, whereby the polymorphic nucleotide in the target nucleic acid is detected.

The invention is also directed to a composition of matter. Here, the invention comprises a metal substrate; a probe oligonucleotide immobilized on the substrate at or near a terminus of the probe oligonucleotide; an upstream oligonucleotide immobilized on the substrate at or near a terminus of the upstream oligonucleotide; and wherein the probe oligonucleotide and the upstream oligonucleotide are immobilized on the substrates at points sufficiently close to one another to allow the probe oligonucleotide and the upstream oligonucleotide to participate cooperatively in an invasive cleavage reaction when the substrate is contacted with a cleavage agent and a target nucleic acid.

The basic parameters of an invasive cleavage assay are presented in U.S. Pat. No. 6,348,314, issued Feb. 19, 2002, and explicitly incorporated herein. See also U.S. Pat. Nos. 6,090,543; 6,001,567; 5,994,069; 5,985,557; and 5,846,717.

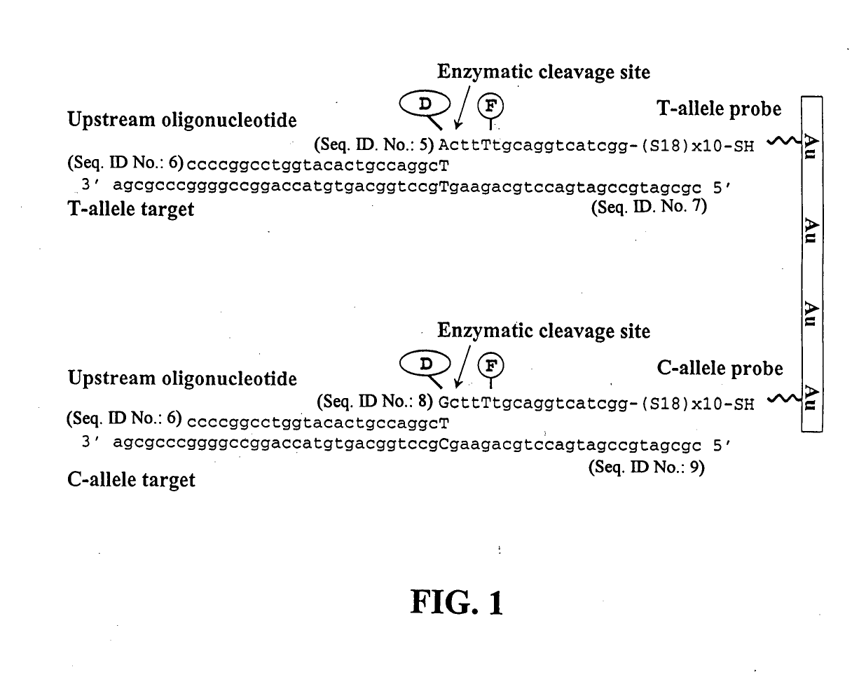

In short, structure-specific oligonucleotide cleavage has been described.^{7, 8, 15} The reaction has high specificity and sensitivity and may be performed in a convenient homogeneous and isothermal biplex format using fluorescence resonance energy transfer (FRET) detection for the simultaneous analysis of both SNP alleles in a single reaction. The specificity results from enzymatic recognition of the structure formed when two separate but overlapping oligonucleotides anneal to a target strand (see FIG. 1). High detection sensitivity stems from the time- and concentration-dependent amplification of signal as probe oligonucleotides are rapidly cycled through the reaction process and are converted to a detectable form. The assay is capable of directly analyzing as few as 600 target molecules⁸ with no requirement for prior PCR amplification.

An upstream oligonucleotide, a probe oligonucleotide, a single-stranded DNA target and a 5′ structure-specific exonuclease form the components of the invasive cleavage reaction. The upstream oligonucleotide hybridizes to the 3′ side of the target and terminates at the base opposite the polymorphism. The probe oligonucleotide is comprised of a 3′ region complementary to the target strand, and a 5′ region that is non-complementary. Hybridization of the probe oligonucleotide to the target yields a duplex with a free unpaired 5′ end or “flap”. A 5′-exonuclease recognizes the structure formed between the upstream oligonucleotide, the probe oligonucleotide, and the target nucleic acid, and cleaves the unpaired flap from the probe oligonucleotide. By operating the assay near the melting temperature of the probe-target duplex, a cycle is formed whereby probe oligonucleotides hybridize to the target, are cleaved by the enzyme, and then melt off of the target. In this way, a single target molecule is capable of assisting in multiple cleavage events. Probe oligonucleotides that are complementary to the target at the position of overlap are cleaved at a rate that is at least 300 times higher than the rate of cleavage for a non-complementary probe.¹⁰ As noted above, this difference in cleavage rate is the basis for the discrimination of single nucleotide differences in the target strand.

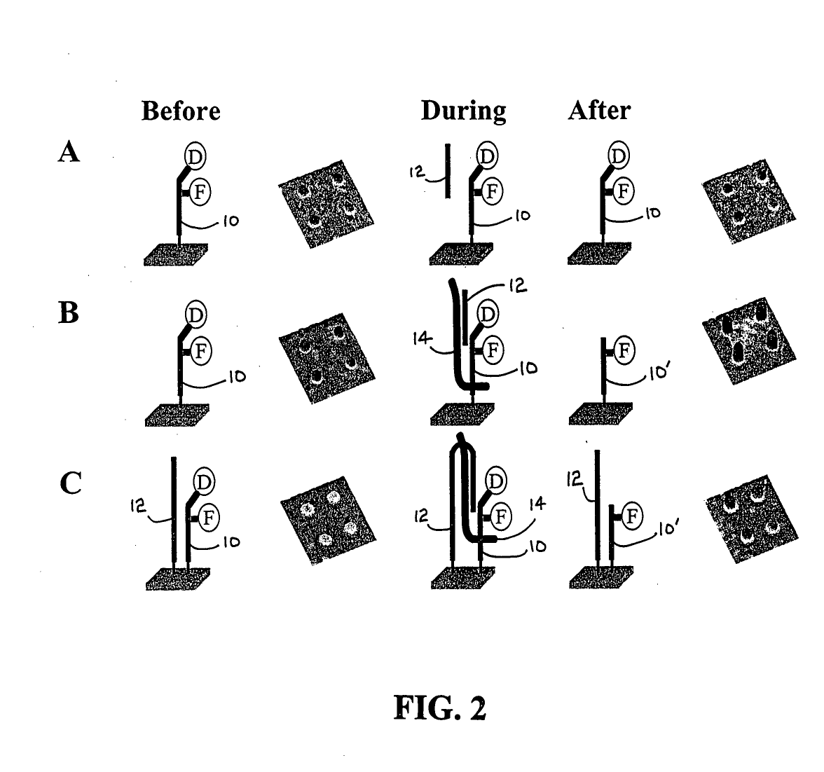

In the present invention, the invasive cleavage reaction is mated to surface-mounting techniques for the parallel analysis of SNPs on a genomic scale. In short, the present invention implements the invasive cleavage assay in a DNA chip format. Using an addressed array of SNP-specific probe oligonucleotides, a single sample of human genomic target DNA added to the surface yields an invasive cleavage structure at every site on the surface that corresponded to a SNP allele in the genome being analyzed. The upstream oligonucleotide, which is also required for the invasive cleavage reaction, can either be added in solution (FIG. 2B, referred to herein as “strategy 1”), or co-immobilized on the surface along with the probe oligonucleotide (FIG. 2C, referred to herein as “strategy 2”). As shown in the Examples, the invasive cleavage reaction gives rise to an increase in fluorescence at that element of the array, indicating the presence of the corresponding SNP allele in the target DNA.

Strategy 2 differs from Strategy 1 in that it obviates the need to add upstream oligonucleotides to the reaction solution. This has the added benefit of eliminating issues associated with non-specific or unintentional interactions between the upstream oligonucleotides. It also makes performance of the reaction much simpler, as only reaction buffer, enzyme, and target DNA need to be added to complete the reaction.

The cleavage agents to be used in the present invention are preferably DNA polymerases that have been modified to render the enzyme polymerase-activity deficient, while retaining the 5′ nuclease activity of the enzyme. Suitable cleavage agents are available commercially from Third Wave Technologies.

Suitable cleavage agents can also be fabricated from known DNA polymerases by methods including, but not limited to: 1) proteolysis; 2) recombinant genetics; and 3) physical and/or chemical modification and/or inhibition.

Proteolysis: Thermostable DNA polymerases having a reduced level of synthetic activity can be produced by physically cleaving the unmodified enzyme with proteolytic enzymes to produce fragments of the enzyme that are deficient in synthetic activity, but retain the desired 5′ nuclease activity. Briefly, following proteolytic digestion, the resulting fragments are separated by standard chromatographic techniques. The separated fragments are then assayed for the ability to synthesize DNA and to act as a 5′ nuclease via means known to the art. (See U.S. Pat. No. 6,348,314.) Recombinant Constructs: U.S. Pat. No. 6,348,314 also describes constructing recombinant constructs that drive the expression of suitable cleavage agents that can be used in the subject invention. In short, the known cloning strategies employed for the Thermus aquaticus and Thermus flavus polymerases are applicable to other thermostable Type A polymerases (due to their close homology). Thus, a thermostable DNA polymerase is cloned by isolating genomic DNA using molecular biological methods from a bacteria containing a thermostable Type A DNA polymerase. This genomic DNA is exposed to primers which are capable of amplifying the polymerase gene by PCR.

This amplified polymerase sequence is then subjected to standard deletion processes to delete the polymerase-encoding portion of the gene. Deletion of amino acids from the protein can be done either by deletion of the encoding genetic material, or by introduction of a translational stop codon by mutation or frame shift.

For example, in the Taq DNA polymerase gene, a deletion between nucleotides 1601 and 2502 (the end of the coding region), a four-nucleotide insertion at position 2043, and deletions between nucleotides 1614 and 1848 and between nucleotides 875 and 1778 will render a quite suitable cleavage agent. (The nucleotide numbering for the Taq gene is that presented in U.S. Pat. No. 6,348,314.) Those skilled in the art understand that single base pair changes can be innocuous in terms of enzyme structure and function. Similarly, small additions and deletions can be present without substantially changing the exonuclease or polymerase function of these enzymes.

Other deletions are also suitable to create the 5′ nucleases of the present invention. It is preferable that the deletion decrease the polymerase activity of the 5′ nucleases to a level at which synthetic activity will not interfere with the use of the 5′ nuclease in the detection assay of the invention. Most preferably, the synthetic ability is absent entirely.

The present invention contemplates that the resulting nucleic acid construct be capable of expression in a suitable host. Those in the art know methods for attaching various promoters and 3′ sequences to a gene structure to achieve efficient expression. Suitable vectors and hosts are described, for example, in U.S. Pat. No. 6,348,314. Of course, there are large number of other promoter/vector combinations that are equally suitable.

Expression can also be accomplished using a cell-free transcription-translation system. Suitable cell-free, in vitro systems are available, such as the “TnT”-brand Coupled Reticulocyte Lysate System (Promega Corporation, Madison, Wis.). Once a suitable nucleic acid construct has been made, the 5′ nuclease may be produced from the construct.

Physical and/or Chemical Modification and/or Inhibition: The DNA synthetic activity of a thermostable DNA polymerase may be reduced by chemical and/or physical means. In one such approach, the cleavage reaction catalyzed by the 5′ nuclease activity of the polymerase is run under conditions which preferentially inhibit the synthetic activity of the polymerase. The level of synthetic activity need only be reduced so that it does not interfere with the cleavage reactions (which do not require significant synthetic activity).

For example, when using Taq DNA polymerases, concentrations of Mg⁺² greater than 5 mM inhibit the polymerization activity of the native enzyme without adversely affecting the 5′ nuclease activity. The ability of the 5′ nuclease to function under conditions where synthetic activity is inhibited is tested by running the assays for synthetic and 5′ nuclease activity, in the presence of a range of Mg⁺² concentrations (e.g., 5 to 10 mM). The effect of a given concentration of Mg⁺² is determined by measuring the amount of synthesis and cleavage in the test reactions as compared to the standard reaction for each assay.

The inhibitory effect of other ions, polyamines, denaturants, such as urea, formamide, dimethylsulfoxide, glycerol and non-ionic detergents (e.g., “Triton X-100”-brand and “Tween-20”-brand detergents), nucleic acid-binding chemicals such as, actinomycin D, ethidium bromide and psoralens, are tested by their addition to the standard reaction buffers for the synthesis and 5′ nuclease assays. Those compounds having a preferential inhibitory effect on the synthetic activity of a thermostable polymerase are then used to create reaction conditions under which 5′ nuclease activity (cleavage) is retained while synthetic activity is reduced or eliminated.

Physical means may also be used to inhibit the synthetic activity of a polymerase. For example, the synthetic activity of thermostable polymerases is destroyed by exposure of the polymerase to extreme heat (typically about 96 to 100° C.) for extended periods of time (greater than about 20 minutes). While there are minor differences with respect to the specific heat tolerance for each type of enzyme, these differences are readily determined. Polymerases are treated with heat for various periods of time and the effect of the heat treatment upon the synthetic and 5′ nuclease activities is determined.

The present invention provides means for detecting single nucleotide polymorphisms (SNPs) by forming a nucleic acid cleavage structure which is dependent upon the presence of a target nucleic acid and then cleaving the nucleic acid cleavage structure so as to release distinctive cleavage products. The activity of a 5′ nuclease is used to cleave the target-dependent cleavage structure and the resulting cleavage products are indicative of the presence of specific target nucleic acid sequences in the sample. The method is run in a heterogeneous format, with at least the probe oligonucleotide immobilized on an inert support. In another embodiment of the invention, both the probe oligonucleotide and the upstream oligonucleotide are immobilized in operationally-connected pairs on the support.

Through the interaction of the cleavage agent (e.g., a 5′ nuclease), and the upstream oligonucleotide, the cleavage agent can be made to cleave a downstream oligonucleotide at a site in such a way that the resulting fragments of the downstream oligonucleotide dissociate from the target nucleic acid, thereby making that region of the target nucleic acid available for hybridization to another, uncleaved copy of the downstream oligonucleotide.

The methods of the present invention employ at least a pair of oligonucleotides that interact with a target nucleic acid to form a cleavage structure for a structure-specific nuclease. The cleavage structure comprises i) a target nucleic acid that may be either single-stranded or double-stranded (when a double-stranded target nucleic acid is employed, it may be rendered single stranded, e.g., by heating); ii) a first oligonucleotide, termed the “probe oligonucleotide,” which defines a first region of the target nucleic acid sequence by being the complement of that region; iii) a second oligonucleotide, termed the “upstream oligonucleotide” or “invader,” the 5′ part of which defines a second region of the same target nucleic acid sequence, adjacent to and downstream of the first target region, and the second part of which overlaps into the region defined by the probe oligonucleotide. (See FIG. 1. and the Examples.)

The upstream oligonucleotide and the probe oligonucleotide are arranged in a parallel orientation relative to one another, while the target nucleic acid strand is arranged in an anti-parallel orientation relative to the upstream and probe oligonucleotides. Using this arrangement, the binding of the probe, upstream, and target oligonucleotides divides the target nucleic acid into three distinct regions: one region that has complementarity to only the probe; one region that has complementarity only to the upstream oligonucleotide; and one region that has complementarity to both the probe and upstream oligonucleotides.

Design of the upstream and probe oligonucleotides is accomplished using practices which are standard in the art. For example, sequences that have self-complementarity, such that the resulting oligonucleotides would either fold upon themselves, or hybridize to each other at the expense of binding to the target nucleic acid, are generally avoided. (This design process is analogous to the process of choosing PCR primers; primers are chosen to avoid or minimize primer dimer formation.)

One consideration in choosing a length for these oligonucleotides is the complexity of the sample containing the target nucleic acid. For example, the human genome is approximately 3×10⁹ base pairs long. Thus, any given 10-nucleotide sequence will appear with a statistical frequency of 1:4¹⁰, or once per 1,048,576 in a random string of nucleotides. This frequency translates to approximately 2,861 appearances of any given 10-nucleotide sequence in a genome of 3 billion base pairs. An oligonucleotide of this length would have a poor chance of binding uniquely to a 10-nucleotide region within a target having a sequence the size of the human genome. In contrast, if the target sequence were within a plasmid of only 3,000 base pairs, such an oligonucleotide might have a very reasonable chance of binding uniquely.

A second consideration in choosing oligonucleotide length is the temperature range in which the oligonucleotides will be expected to function. A 16-mer of average base content (50% G-C base pairs) will have a calculated Tₘ (the temperature at which 50% of the sequence is dissociated) of about 41° C., depending on, among other things, the concentration of the oligonucleotide and its target, the salt content of the reaction and the precise order of the nucleotides. As a practical matter, longer oligonucleotides are usually chosen to enhance the specificity of hybridization. Oligonucleotides 20 to 25 nucleotides in length are often used as they are highly likely to be specific if used in reactions conducted at temperatures that are within about 5° C. from their Tm's. In addition, 20- to 25-mers with calculated Tm's in the range of 50° C. to 70° C. are appropriately used in reactions catalyzed by thermostable enzymes, which often display optimal activity near this temperature range.

The maximum length of the oligonucleotide chosen is also based on the desired specificity. Choosing sequences that are so long that they are either at a high risk of binding stably to partial complements, or that they cannot easily be dislodged when desired (e.g., failure to disassociate from the target once cleavage has occurred) should be avoided.

The first step of design and selection of the oligonucleotides for the invasive cleavage is in accordance with these general principles. Thus, each oligonucleotide will generally be long enough to be reasonably expected to hybridize only to the intended target sequence within a complex sample, usually in the 20 to 40 nucleotide range. Alternatively, because the invasive cleavage assay depends upon the concerted action of these oligonucleotides, the composite length of the probe and upstream oligonucleotides may be selected to fall within this range, with each of the individual oligonucleotides being in the roughly 13 to 17 nucleotide range. Such a design might be employed if a non-thermostable cleavage means were employed in the reaction. A non-thermostable cleavage agent requires the reactions to be conducted at a lower temperature than that used when thermostable cleavage means are employed. In some instances, it may be desirable to have these oligonucleotides bind multiple times within a target nucleic acid (e.g., which bind to multiple variants or multiple similar sequences within a target). It is not intended that the method of the present invention be limited to any particular size of the probe or upstream oligonucleotide.

The second step of designing an oligonucleotide pair for this assay is to choose the degree to which the upstream “invader” oligonucleotide sequence will overlap into the downstream “probe” oligonucleotide sequence, and consequently, the sizes into which the probe will be cleaved. For detection of a SNP, a single base pair overlap is desired. (See FIG. 1.)

Target nucleic acids that can be analyzed using the present invention include both RNA and DNA. The target nucleic acid may be obtained using standard molecular biological techniques. For example, nucleic acids may be isolated from tissue samples, tissue culture cells, samples containing bacteria and/or viruses (including cultures of bacteria and/or viruses), etc. The target nucleic acid may also be transcribed in vitro from a DNA template or may be chemically synthesized or generated in a PCR protocol. Furthermore, nucleic acids may be isolated from an organism, either as genomic material or as a plasmid or similar extrachromosomal DNA, or they may be a fragment of such material generated by treatment with a restriction endonuclease or other cleavage agents or they may be wholly synthetic (e.g., a synthetic combinatorial library of polynucleotides).

Assembly of the target, probe, and upstream nucleic acids into the cleavage reaction of the present invention uses principles commonly used in the design of oligonucleotide base enzymatic assays, such as dideoxynucleotide sequencing and polymerase chain reaction (PCR). As is done in these assays, the oligonucleotides are provided in sufficient excess that the rate of hybridization to the target nucleic acid is very rapid. These assays are commonly performed with 50 fmoles to 2 pmoles of each oligonucleotide per μl of reaction mixture. The concentration of probe and/or upstream oligonucleotide immobilized on the inert substrate is controlled during the immobilization process. Contacting the substrate with probe solutions of greater concentration or for longer reaction times generally results in chips having greater probe density.

It is desirable that upstream oligonucleotide be immediately available to direct the cleavage of each probe oligonucleotide that hybridizes to a target nucleic acid. For this reason, when it is present in solution, the upstream oligonucleotide is provided in excess as compared ot the probe oligonucleotide. As a general rule, this excess is about 10-fold. While this is an effective ratio, it is not intended that the practice of the present invention be limited to any particular ratio of upstream-to-probe (a ratio of about 2- to about 100-fold is contemplated).

Buffer conditions must be chosen that will be compatible with both the oligonucleotide/target hybridization and with the activity of the cleavage agent. The optimal buffer conditions for nucleic acid-modification enzymes, and particularly DNA modification-enzymes, generally included enough mono- and divalent salts to allow association of nucleic acid strands by base-pairing. If the method of the present invention is performed using an enzymatic cleavage agent other than those specifically described herein, the reactions may generally be performed in any such buffer reported to be optimal for the nuclease function of the cleavage agent chosen. In general, to test the utility of any cleavage agent in this method, test reactions are performed wherein the cleavage agent of interest is tested in the MOPS/MnCl₂/KCl buffer or Mg-containing buffers described herein and in whatever buffer has been reported to be suitable for use with that agent.

The products of the cleavage reaction are fragments generated by structure-specific cleavage of the input oligonucleotides. The resulting cleaved and/or uncleaved oligonucleotides may be analyzed and resolved by a number of methods including including FRET detection, which is preferred. See the Examples for a further discussion.

Alternatively, the probe and/or invader oligonucleotides may contain a label to aid in their detection following the cleavage reaction. The label may be a radioisotope (e.g., a ³²P or ³⁵S-labelled nucleotide placed at either the 5′ or 3′ end of one of the oligonucleotides. The label might also be distributed throughout the oligonucleotide (i.e., a uniformly labelled oligonucleotide). The label may be a nonisotopic detectable moiety, such as a fluorophore, which can be detected directly, or a reactive group which permits specific recognition by a secondary agent. For example, biotinylated oligonucleotides may be detected by probing with a streptavidin molecule which is coupled to an indicator (e.g., alkaline phosphatase or a fluorophore) or a hapten such as dioxigenin may be detected using a specific antibody coupled to a similar indicator. Generally, a preferred route takes advantage of the chip format of the invention, and thus “reads” and “scores” the chip automatically, using known spectrophotometric means and equipment (e.g., “plate readers” with associated optics and data-handing sub-assemblies, the optics including band-pass filters and the like to discriminate between background noise and signal).

The cleavage reaction is useful to detect the presence of specific nucleic acids. In addition to the considerations listed above for the selection and design of the invader and probe oligonucleotides, the conditions under which the reaction is to be performed may be optimized for detection of a desired target sequence.

One objective in optimizing the cleavage reaction is to allow specific detection of the fewest copies of a target nucleic acid. To achieve this end, it is desirable that the combined elements of the reaction interact with the maximum efficiency, so that the rate of the reaction (e.g., the number of cleavage events per minute) is maximized. Elements contributing to the overall efficiency of the reaction include the rate of hybridization, the rate of cleavage, and the efficiency of the release of the cleaved probe.

The rate of cleavage will be a function of the cleavage means chosen, and may be made optimal according to the manufacturer's instructions when using commercial preparations of enzymes. The other elements (rate of hybridization, efficiency of release) depend upon the execution of the reaction, and optimization of these elements is discussed below.

Three elements of the cleavage reaction that significantly affect the rate of nucleic acid hybridization are the concentration of the nucleic acids, the temperature at which the cleavage reaction is performed, and the concentration of salts and/or other charge-shielding ions in the reaction solution.

The concentrations at which oligonucleotide probes are used in assays of this type are well known in the art, and are discussed above and in the Examples. One example of a common approach to optimizing an oligonucleotide concentration is to choose a starting amount of oligonucleotide for pilot tests. When these initial cleavage reactions are performed, the following reactions can be assembled to systematically optimize the reaction conditions: 1) Perform the reaction in the absence of the target nucleic acid and determine if the reaction products are substantially free of the cleavage product. 2) Perform the reaction with systematically-modified upstream oligonucleotides, using a known probe and known target. Then determine if the site of cleavage is specifically shifted in accordance with the design of the upstream oligonucleotide. 3) Run the reaction at serially-diluted concentrations of target to determine if the specific cleavage product can easily be distinguished from the uncleaved probe.

If the above test runs provide unsatisfactory results, the probe concentration is likely too high. A set of reactions using serial dilutions of the probe (i.e., using chips of decreasing probe density) should be performed until the appropriate probe density is identified. Once identified for a given target nucleic acid in a give sample type (e.g., purified genomic DNA, body fluid extract, lysed bacterial extract), it should not need to be re-optimized. The sample type is important because the complexity of the material present may influence the probe density optimum.

Conversely, if the chosen initial probe concentration (density) is too low, the reaction may be slow due to inefficient hybridization. Tests with increasing density of the probe (or upstream oligo) will identify the point at which the concentration exceeds the optimum. Because the hybridization will be facilitated by excess of probe, it is desirable, but not required, that the reaction be performed using probe densities just below this point.

The concentration (or density) of the upstream oligonucleotide can be chosen based on the design considerations discussed above. In a preferred embodiment, the invader oligonucleotide is in excess of the probe oligonucleotide. In a particularly preferred embodiment, the invader is approximately 10-fold more abundant than the probe.

Temperature is also an important factor in the hybridization of oligonucleotides. The range of temperature tested will depend in large part on the design of the oligonucleotides. Generally, the reactions are performed at temperatures slightly below the Tm of the least stable oligonucleotide (i.e., lowest Tm oligo) in the reaction. Melting temperatures for the oligonucleotides and for their component regions can be estimated through the use of computer software or, for a more rough approximation, by assigning the value of 2° C. per A-T base pair, and 4° C. per G-C base pair, and taking the sum across the expanse of oligonucleotide. (This rule of thumb give a good approximation of Tm for oligonucleotides of approximately 10-30 nucleotides in length.) Because even computer-assisted predictions of the Tm of a nucleic acid are only approximations, the reaction temperatures chosen for initial tests should bracket the calculated Tm.

When temperatures are tested, the results can be analyzed for specificity in the same way as for the oligonucleotide concentration determinations. Non-specific cleavage indicates non-specific interactions between the probe and the target material, and generally suggests that a higher temperature should be employed. Conversely, little or no cleavage in the presence of target suggests that even the intended hybridization is being prevented. Lower reaction temperatures are indicated in this instance. By testing several temperatures it is possible to identify an approximate temperature optimum, at which the rate of specific cleavage of the probe is highest, while non-specific cleavage is lowest.

A third determinant of hybridization efficiency is the salt concentration of the reaction. In large part, the choice of solution conditions will depend on the requirements of the cleavage agent, and for reagents obtained commercially, the manufacturer's instructions are a resource for this information. When developing an assay utilizing any particular cleavage agent, the oligonucleotide and temperature optimizations described above should be performed in the buffer conditions best suited to that cleavage agent.

### EXAMPLES

The following Examples are included solely to provide a more complete and consistent understanding of the invention disclosed and claimed herein. The Examples do not limit the scope of the invention in any fashion.

Sequence Design:

A polymorphic site in codon 158 of the human ApoE gene was used as a model system to test the surface invasive cleavage reaction. A pair of probe oligonucleotides, differing only at the polymorphic nucleotide (“T-allele probe” and “C-allele probe”), one upstream oligonucleotide, and two synthetic targets (“T-allele target” and “C-allele target”) were designed to meet the normal requirements for an invasive cleavage reaction (see FIG. 1). A dabcyl-fluorescein FRET pair (“D” and “F” in FIG. 1) is incorporated into the probe oligonucleotide sequence with dabcyl, the quencher, at the 5′ end of the probe. The 3′ end of the probe contains a free thiol group for covalent coupling to a maleimide group present on the surface followed by a series of ten 18-atom spacer moieties, providing a total spacer length of 240 angstroms. The use of such a spacer region between an oligonucleotide and a surface is often critical to obtaining good performance in surface hybridization.²¹

Oligonucleotide Synthesis:

All unmodified oligonucleotides, including the upstream oligonucleotide, target strands, and PCR primers (see the following section) were obtained (PAGE-purified) from Integrated DNA Technologies (Coralville, Iowa). The surface-bound FRET probe oligonucleotides and upstream oligonucleotide (FIG. 1) were obtained from Third Wave Technologies (Madison, Wis.). The surface-bound cleaved probe (5′-ctt-(fluorescein-dT)-tgcaggtcatcgg (spacer phosphoramidite 18)₁₀-SH-3′) (SEQ. ID. NO: 1) was synthesized at the University of Wisconsin Biotechnology Center (Madison, Wis.). The 5′ dabcyl phosphoramidite, fluorescein-dT, spacer phosphoramidite 18, and 3′-thiol modifier C3 S-S CPG500 used in the synthesis were all purchased from Glen Research (Sterling, Va.). Prior to purification, both 3′ and 5′ thiol-modified oligonucleotides were deprotected as outlined by Glen Research Corp.²² The oligonucleotides containing free thiol groups were then purified by reverse-phase binary gradient elution HPLC (Shimadzu SCL-6A), and stored under an inert atmosphere. Oligonucleotide concentrations were determined by measuring absorption at 260 nm with an HP8453 UV-VIS spectrophotometer.

DNA Surface Attachment Chemistry:

The thiol-modified oligonucleotides were immobilized on gold thin films via a four-step chemical modification described elsewhere.^{23, 24} In brief, a self-assembled monolayer of the alkanethiol, 11-mercaptoundecanoic acid (MUA) (Aldrich) was formed on a gold-coated glass substrate (Evaporated Metal Films, NY), followed by electrostatic adsorption of a poly-L-lysine (PL) (Sigma) monolayer through the carboxylic acid groups of MUA and the amine groups of PL. Free amine groups on PL not involved in the electrostatic interaction with the acid-terminated surface were then reacted with the heterobifunctional linker sulfosuccinimidyl 4-(N-maleimidomethyl) cyclohexane-1-carboxylate (SSMCC) (Pierce), creating a thiol-reactive, maleimide-terminated surface that can covalently interact with thiol-modified DNA strands. For the probe-only immobilization strategy, 0.5 μL of 0.8 mM thiol-modified probe oligonucleotide was deposited at discrete locations on this maleimide-terminated surface. For the co-immobilization strategy, 0.5 μL aliquots containing both 0.4 mM thiol-modified probe oligonucleotide and 0.4 mM thiol-modified upstream oligonucleotide were mixed first and deposited. The surface attachment reaction was permitted to run for approximately 20 hours in a humid chamber to prevent evaporation. Afterward, the surface was rinsed with distilled water and soaked in 10 mM 4-morpholinepropanesulfonic acid (MOPS)/7.5 mM MgCl₂ (pH 7.5), the invasive cleavage reaction buffer, at 60° C. for 3 hours to remove nonspecifically bound DNA.²³

An alternative method of DNA attachment was used for the allelic discrimination experiment. This approach used PCR-amplified target DNA. Thiol-modified probe oligonucleotides were linked via SSMCC to an amine-terminated alkanethiol 11-mercaptoundecylamine (MUAM) (Dojindo Laboratories, Japan) modified gold substrate. The covalent bonds between the layers of the chemical linkers created a more stable surface.²⁵

The Surface Invasive Cleavage Reaction:

The 200 μL reaction solution contained: 10 mM MOPS (pH 7.5), 7.5 mM MgCl₂, 0.25 μM upstream oligonucleotide in the case of probe-only immobilization strategy, 1000 ng Afu FEN 1 (commercially available in the Factor V Leiden RUO Kit from Third Wave Technologies, Madison, Wis.), and 50 μM to 5 nM synthetic target DNA or single-stranded PCR product. The gold surfaces were fully covered by the 200 μL reaction mix, and incubated at a temperature between 52 and 61° C. for up to 24 hours in a humid chamber. The surface fluorescence was measured with a FluorImager 575 (Molecular Dynamics, Sunnyvale, Calif.) both before and after reaction. DNA Amplification, Strand Separation and Quantification:

A set of PCR primers, 5′-biotin-acagaattcgccccggcctggtacactgcca-3′ (SEQ. ID. NO: 2) and 5′-tccaaggagctgcaggcggcgca-3′ (SEQ. ID. NO: 3), yielded a 228 nucleotide (nt) fragment containing codon 158 of the human ApoE gene. The 25 μL amplification reaction mixture contained 10% DMSO (Sigma), 1×PCR buffer, 2 mM MgCl₂, 200 μM each dATP, dCTP, dTTP, and dGTP, 2.5 U AmpliTaq DNA Polymerase (Applied Biosystems, California), 1 μM each primer, and 100 ng genomic DNA sample (provided by Third Wave Technology). The PCR reactions were performed on a PTC-200 Peltier Thermal Cycler (MJ Research, Waltham, Mass.) using the following program: denaturation at 94° C. for 2 min, 40 PCR cycles of denaturation at 94° C. for 30 sec, annealing at 65° C. for 30 sec, and extension at 72° C. for 45 sec, with the final cycle extension running for 10 min.

The PCR mixture was purified using the High Pure PCR Product Purification Kit (Roche Molecular Biochemicals). Strand separation of the PCR product was accomplished using streptavidin-coated magnetic beads (Dynabeads M-280, Dynal, Great Neck, N.Y.). Beads (1 mg) were prewashed with PBS, pH 7.4 (GIBCO BRL, Grand Island, N.Y.) containing 0.1% bovine serum albumin (Sigma), and 1×B&W buffer (5 mM Tris-HCl, pH 7.5, 0.5 mM EDTA, 1.0 mM NaCl). Biotinylated PCR product (100 mL) was added to the streptavidin beads along with 100 μL 2×B&W buffer. The mixture was incubated at room temperature for 15 min with frequent shaking. The beads were then washed twice in 1×B&W buffer before addition of 100 PL 0.1 N NaOH to separate the double-stranded PCR product bound to the beads. The denaturation reaction was kept at room temperature for 10 min. The supernatant containing the non-biotinylated DNA strand was collected and then neutralized with 10 μL 1 M HCl. The single-stranded DNA was purified with Microcon 50 (Amicon, Beverly, Mass.) to remove EDTA and excess salt before use in the invasive cleavage reaction.

To quantify the amount of PCR amplicon used in the reaction, the primer corresponding to the final single-stranded PCR product was modified with fluorescein as 5′-fluorescein-tccaaggagctgcaggcggcgca-3′ (SEQ. ID. NO: 4). Following the strand separation described above, the fluorescein-tagged, single-stranded PCR product was collected, and the fluorescence emission of the sample at 520 nm (excitation wavelength of 497 nm) was measured with a Hitachi F-4500 fluorescence spectrophotometer. The amount of this unknown sample was estimated to be approximately 0.5 pmole by reference to a standard curve prepared from a series of known fluorescent samples.

Simulation of the Progress of the Surface Cleavage Reaction:

In order to relate the observed changes in surface fluorescence intensity to the progress of the surface invasive cleavage reaction, two oligonucleotides, the surface-bound FRET probe oligonucleotide (containing both fluorescein and dabcyl, and hence quenched) and the cleaved probe oligonucleotide (containing only fluorescein, and hence unquenched), were mixed in ratios of 5:0, 4:1, 3:2, 2:3, 1:4 and 0:5, corresponding to probe cleavage fractions of 0, 20, 40, 60, 80 and 100%, before deposition onto the surface in quadruplicate. The total concentration of the two oligonucleotides in the mixture was kept at 0.8 mM, consistent with the conditions under which the surface invasive cleavage reaction was performed in a probe-only immobilization format. The fluorescence intensity of each spot on the surface was measured with the FluorImager 575. A plot of the signal increase as a function of the probe cleavage fraction was made using the average fluorescence intensity of each quadruplicate sample.

Results of the Examples and Their Significance:

Two Curface Invasive Cleavage Reaction Strategies:

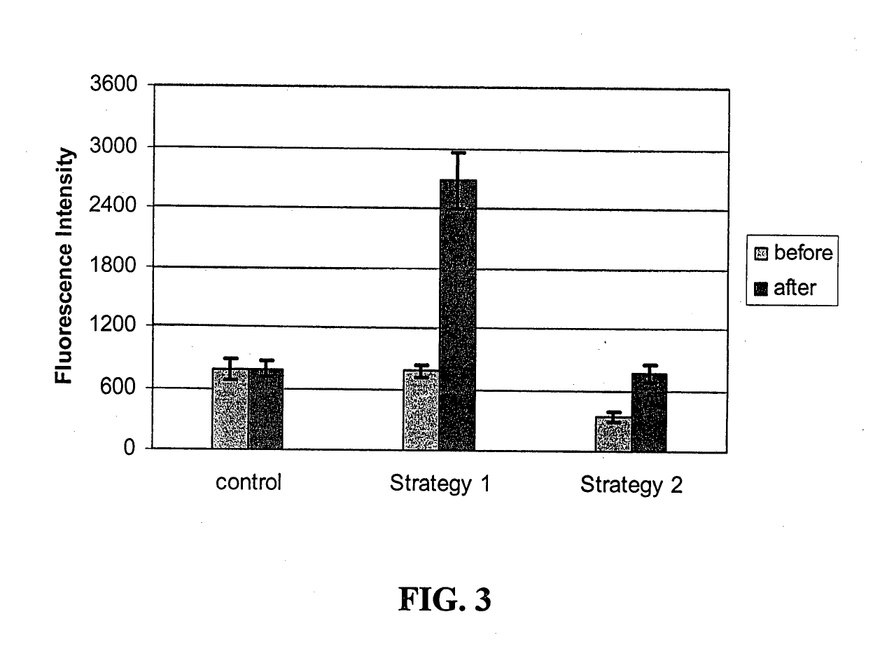

FIGS. 2A, 2B, 2C, and 3 show the results of experiments in which the invasive cleavage reaction was performed on oligonucleotide-immobilized planar gold substrates using 50 pM synthetic t-allele target and a 24-hour reaction time. The t-allele probe, along with upstream oligonucleotide in a 1:1 molar ratio for the case of co-immobilization, was coupled to an 18×18 mm gold surface to form a 2×2 array of 2 mm diameter spots. In FIG. 2B, where only the probe oligonucleotide was immobilized on the surface, but the upstream oligonucleotide was added in solution with target strand, the fluorescence intensity increased by a factor of 3.5 (average of the four spots) after the invasive cleavage reaction. In FIG. 2C, where both of the probe and upstream oligonucleotides were immobilized on the surface and only the target strand was added in solution, a 2.3-fold increase in fluorescence intensity was produced. In both cases, a significant increase in fluorescence signal was observed in the presence of the target molecules, but was not observed in the control experiment with no added target (FIG. 2A), demonstrating the target-dependent specificity of the reaction.

The fundamental source of the initial intensity of the probe oligonucleotide is residual fluorescence from incompletely quenched fluorophore donor. After subtraction of the background signal from the surface, the initial fluorescence signal on the co-immobilized surface (332 RFU) is lower than that on the probe-only immobilized surface (772 RFU) (see FIG. 3). On the assumption that the total oligonucleotide surface density under the experimental conditions employed remains constant, this lower initial fluorescence signal can be attributed to the two-fold lower surface probe density resulting from the dilution of the co-immobilized upstream oligonucleotides at a 1:1 molar ratio. This lower surface probe density on the co-immobilized surface also yielded considerably less signal increase (430 RFU vs. 1910 RFU on the probe-only immobilized surface, see FIG. 3) as the signal generation in the invasive cleavage reaction is directly associated with the amount of probe oligonucleotide.¹⁰

Although the co-immobilization strategy yields a lower increase in fluorescence signal intensity, it is a more practical format for large-scale genotyping on DNA arrays. There are several reasons for this: first, it would likely be problematic to have a sufficient concentration of hundreds of thousands of different upstream oligonucleotides in solution at one time; second, it is likely that interactions between these strands would occur, which would compromise their ability to function in the surface cleavage reaction; third, it would introduce the issue of having to synthesize and dispense hundreds of thousands of individual chemical reagents (along with having to attend to the required quality control issues). Whereas if the DNA molecules were all synthesized in situ on the support, as is done in existing oligonucleotide array fabrication, these issues do not exist. Finally, having the upstream oligonucleotide already in close proximity to its companion probe oligonucleotide on the support may provide advantages in the formation of the necessary quaternary complex required for the invasive cleavage reaction.

SNP Analysis Using the Surface Invasive Cleavage Reaction:

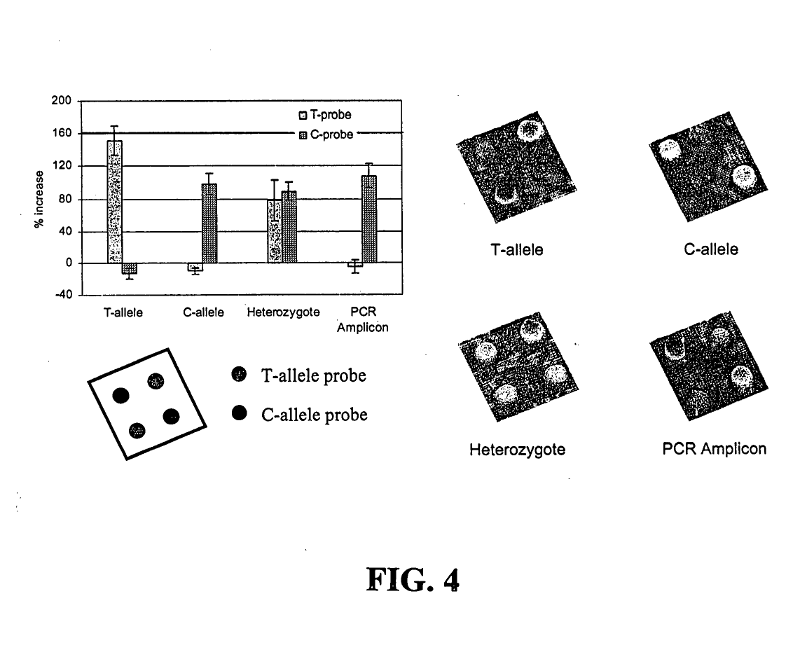

FIG. 4 shows the results obtained using surfaces to which both the c-allele probe and the t-allele probe were attached, and the target employed was a single stranded PCR amplicon from the human ApoE gene generated from a human genomic DNA sample. Although it is simpler to prepare double-stranded PCR products than single-stranded, hybridization of the double-stranded molecule to the surface will necessarily suffer from competition of the complementary strand with the surface-bound probe oligonucleotide. Previous work has shown that the surface hybridization efficiency is substantially higher with the single-stranded than the double-stranded product.²¹ Therefore, single-stranded PCR amplicon was used exclusively in the surface invasive cleavage reaction experiments.

Control experiments were performed with synthetic targets corresponding to the c-allele, the t-allele, or a 1:1 mixture of both, representing a heterozygous genotype. In each case the appropriate results were observed. The homozygous t-allele or c-allele targets yielded fluorescence signal increase only for the corresponding probe oligonucleotide, t-allele probe or c-allele probe, respectively. In contrast, the mock heterozygous sample generated similar signal increases for both of the probe oligonucleotides. The PCR amplicon target resulted in increased surface fluorescence only for the c-allele probe, indicating a homozygous c-allele genotype for the individual in question. This result is consistent with the result obtained using a standard solution invasive cleavage reaction. These results demonstrate the formation of the invasive cleavage structure on the surface and its specific recognition and cleavage by the Afu FEN enzyme, with single nucleotide specificity. In addition, the ability to employ a PCR amplicon as target demonstrates the feasibility of SNP genotyping on surfaces from genomic DNA samples.

It may be noted that the signal increase is not uniform for the two probe oligonucleotides under the same reaction conditions. It has been shown in a theoretical analysis of the solution-phase invasive cleavage reaction that the exchange of the probe oligonucleotide on and off the target strand is the rate-limiting kinetic step of the reaction.¹⁰ The generation of multiple cleavage events per target molecule is achieved by operating the reaction near the Tm of the probe-target duplex, where the cleaved probe is readily melted off from the target strand, and replaced by an uncleaved one. Therefore, for a given set of upstream, probe and target oligonucleotides, reaction buffer conditions and enzyme concentration, there is an optimum temperature for maximum signal amplification. A higher temperature would result in unstable hybridization between the probe and the target, and a lower temperature would inhibit the cycling of the probe oligonucleotide. Both scenarios result in a lower amount of cleavage. The two probe oligonucleotides used in the Examples have different Tms due to the sequence difference at the polymorphic site (t vs. c). Therefore, one likely reason for the observed difference in signal generation for the two probe oligonucleotides on the surface is the difference in their Tms. This difference can be minimized, if desired, by varying the length and/or composition of the probe oligonucleotides to yield similar Tms. A preliminary investigation of this temperature issue for the surface invasive cleavage reaction on the probe-only surfaces will be discussed below. Another possible reason for this difference in signal generation is variability in the surface density of the two probe oligonucleotides resulting from differences in the self-assembled monolayer, the layers of the chemical linkers, and/or the coupling efficiency of the probes on each gold slide. Such surface variability is not expected to be large as all the surfaces and oligonucleotides employed were prepared at the same time and under similar conditions.

Optimum Reaction Temperature:

The effect of reaction temperature on the surface invasive cleavage reactions was investigated on surface arrays of t-allele probes using 5 nM synthetic t-allele target with a reaction time of 3 hours. Varying the temperature from 52 to 61° C. showed that the greatest increase in fluorescence intensity occurred at approximately 54° C. Interestingly, this optimum temperature of 54° C. at which the surface invasive cleavage reaction proceeds at a maximum rate is significantly different than the optimum temperature of 60° C. observed in solution experiments with the same sequences (data not shown). A similar decrease in the optimum temperature was observed with oligonucleotides immobilized on latex microparticles.¹⁸

As discussed above, the optimum temperature in the solution-phase invasive cleavage reactions is near the melting temperature (Tm) of the probe-target duplex structure.¹⁰ The reduced optimum temperature observed on surfaces, therefore, might indicate a lower Tm for the surface hybridization than for the corresponding solution hybridization. The Tm is known to depend strongly upon the concentration of the DNA strands,²⁶ and in the case of the surface invasive cleavage experiments the “concentration” of the surface-immobilized probe is quite low, limited by the amount of surface area available and the surface density of the oligonucleotides of ˜5×10¹² molecules/cm.²³⁻²⁵ The total amount of probe oligonucleotide available in the four 2 mm-diameter spots on the surface is approximately 1 pmole. However, the 2-dimensional surface system makes the definition of DNA “concentration” complicated because the attached probe oligonucleotides are no longer uniformly distributed as they are in a 3-dimensional solution. A very simplistic approach to this problem would be to neglect this surface effect, and calculate “effective” concentrations as if the probe oligonucleotides were uniformly dispersed in the entire experimental solution volume. For the 200 μL volume employed here, this yields an “effective” probe concentration of 5 nM, compared to the typical probe concentration employed in solution-phase invasive cleavage reactions of 500 nM. Using the nearest-neighbor model,^{27, 28} estimates of the expected difference in Tm for the solution and surface experiments, based upon these differences in probe “concentration” in the two experiments, yield a predicted Tm that is 7.5° C. lower for the surface cleavage reaction than for the solution reaction. Solution-phase temperature titration experiments using the lower probe concentration, 5 nM, also generated an optimum temperature that is 6.6° C. lower (data not shown). Both of these results are comparable to the observed decrease of 6° C. on surface. Thus one likely explanation for the observed difference in optimum temperature is that it is a direct consequence of the relatively low numbers of probe molecules participating in the reaction in the surface experiments. Other possible explanations include electrostatic effects of the surface upon DNA or enzyme binding,²⁹ and steric or other effects of the surface upon the kinetics of the DNA hybridization or enzymatic cleavage reactions.

Fluorescence as a Function of Cleavage Fraction:

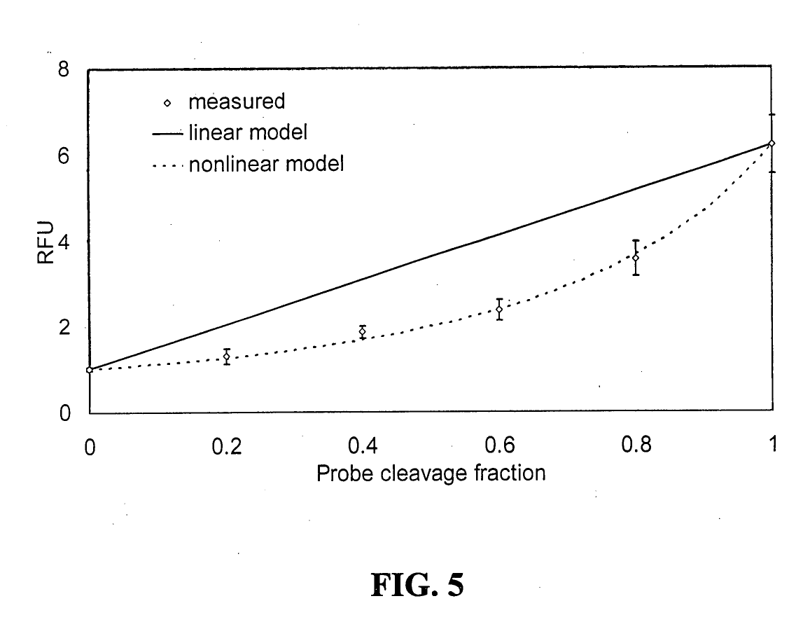

In order to study the underlying mechanism of the surface cleavage reaction, it is essential to be able to determine the fraction of probe molecules that are cleaved on the surface under a given set of conditions. The most straightforward approach to obtain such information is to monitor the changes in surface fluorescence intensity during the course of the cleavage reaction. This requires, however, that the relationship between surface fluorescence intensity and the fraction of cleaved probes on the surface be known. To evaluate this relationship surfaces were prepared with varying proportions of cleaved and uncleaved probe oligonucleotides (in the same fashion as described in the previous Examples), and the surface fluorescence intensity was measured for each sample. The results of this Example are shown in FIG. 5.

A very useful and important parameter in describing FRET on surfaces is the energy transfer efficiency, E, which can be readily obtained from the data of FIG. 5. In the same fashion as is in solution, E is defined as³⁰

$$E = 1 - \frac{{I}_{FQ}}{{I}_{F}} \qquad ( 1 )$$

Here, I_{FQ} and I_{F} denote the fluorescence intensities of the quenched and non-quenched probes, respectively. The fluorescence intensities shown in FIG. 5 were normalized to the intensity measured at a probe cleavage fraction of 0, giving a value for I_{FQ}=1.0. The fluorescence intensity at a probe cleavage fraction of 1 (complete probe cleavage) provides the other limit, corresponding to I_{F}=6.23. Using these two values E is readily calculated to have a value of 0.84. Interestingly, this surface efficiency is lower than that observed in solution with the same probe oligonucleotides (Eₛₒₗᵤₜᵢₒₙ˜0.91, data not shown). A possible explanation for this less efficient FRET process on the surface is that the attachment of the oligonucleotide onto the surface restricts its conformational flexibility, and that this reduced flexibility reduces the efficiency of the dipole-dipole interaction between the dye and quencher molecules that mediates the FRET process. Applicants, however, are not limited by this interpretation of the underlying phenomena that give rise to this observation.

An interesting aspect of the experimental results shown in FIG. 5 is the nonlinear relationship between the surface fluorescence intensity and the fraction of cleaved probe. If it is assumed that the FRET process is restricted to interactions between the fluorescence donor and acceptor on the same probe, and that the corresponding energy transfer efficiency is a constant during the reaction, the fluorescence intensity is expected to be a linear function of the probe cleavage fraction. This function would be given by the sum of the fluorescence contributions of the two populations of molecules on the surface, as follows:

$$\begin{matrix}I ( x ) = {xI}_{F} + ( 1 - x ) {I}_{FQ} \\ = x \times 6.23 + ( 1 - x ) \times 1 \\ = 5.23 x + 1\end{matrix} \qquad ( 2 )$$

where x corresponds to the probe cleavage fraction, and I(x) is the total fluorescence intensity observed.

However, as shown in FIG. 5, at each measured probe cleavage fraction between 0 and 1, the observed fluorescence intensity is lower than that predicted by this linear relationship. To explain this behavior, it was hypothesized that in addition to the intramolecular energy transfer process, there might be fluorescence quenching effects occurring between adjacent probe oligonucleotides on the surface. From the surface density of approximately 5×10¹² molecules per cm², the average distance between two adjacent probe molecules may be estimated to be about 50 Å. As the energy transfer efficiency of FRET is known to be inversely proportional to the sixth power of the distance between the donor and accepter dye molecules, and is generally effective within the range of 10 and 100 Å,³¹⁻³⁴ this mechanism seemed to be a likely possibility. The prediction of this hypothesized mechanism is qualitatively in accord with the observations. Thus, at low probe cleavage fractions with a large number of dabcyl quenchers on the surface, the emission from fluorescein on the cleaved probe is substantially suppressed by the intermolecular quenching effect. However, the effect becomes less significant as the probe cleavage fraction increases, because the density of quenchers on the surface decreases and thus the intermolecular quenching process becomes less efficient. Therefore a greater increase in fluorescence signal is observed at higher cleavage fractions than would be predicted by the linear model.

To provide a quantitative description of this hypothesis, a simple mathematical model that relates the steady-state fluorescence signal measured from the surface to the progress of the invasive cleavage reaction is presented. In establishing this model, the goal was to achieve a high level of fitness using a minimum number of assumptions. For convenience, the oligonucleotides attached on the surface are assumed to be assembled in a hexagonal close-packed monolayer with a spacing of approximately 50 Å. As discussed previously, the signal generated from the surface is divided into two parts corresponding to the state of the probe molecule. Signal from intact probe molecules having both a fluorophore and a quencher is denoted I_{FQ} and signal from cleaved probe molecules with only a fluorophore is IF.
I(x)=xI_{F}(x)+(1−x)I_{FQ} (3).

Furthermore, due to the proximity of quencher and fluorophore on intact probe molecules, intra-molecular quenching is the dominant form of energy transfer. Interactions between fluorophores on intact probes with other molecules are therefore ignored and the total contribution to the measured intensity of the intact probes is taken to be proportional to the fraction of cleaved probes (1−x). Explaining the contribution to the measured intensity from cleaved probe molecules, I_{F}(x), requires a more detailed analysis.

First, the probability that any fluorophore is excited at time t, P_{F}(t), is examined. The time derivative has the following form.

$${\dot{P}}_{F} ( t ) = - \frac{1}{τ} {P}_{F} ( t ) - {\sum }_{i} {k}_{i} {P}_{F} ( t ) + c \qquad ( 4 )$$

This equation takes into account the fluorescence decay

$$- \frac{1}{τ} {P}_{F} ( t )$$

where τ is the fluorescence lifetime, quenching/energy transfer

$$- {\sum }_{i} {k}_{i} {P}_{F} ( t )$$

where kᵢ is the rate constant for quenching by the i^{th} quencher, and steady-state pumping c. At steady state, \dot{P}_{F}(t)=0, making

$${P}_{F} ( \frac{1}{τ} + {\sum }_{i} {k}_{i} ) = c \qquad ( 5 )$$

Averaging over all possible configurations of quenchers gives

$$〈 {P}_{F} {\sum }_{i} {k}_{i} 〉 \approx 〈 {P}_{F} 〉 〈 {\sum }_{i} {k}_{i} 〉 \qquad ( 6 )$$

and therefore

$$〈 {P}_{F} 〉 ( \frac{1}{τ} + 〈 {\sum }_{i} {k}_{i} 〉 ) = c \qquad ( 7 )$$

or upon rearrangement

$$〈 {P}_{F} 〉 = \frac{c}{\frac{1}{τ} + 〈 {\sum }_{i} {k}_{i} 〉} = \frac{c τ}{1 + τ 〈 {\sum }_{i} {k}_{i} 〉} \qquad ( 8 )$$

For probes on a hexagonal lattice

$$τ 〈 {\sum }_{i} {k}_{i} 〉 = 6 τ 〈 {k}_{i} 〉 = 6 τ \frac{1}{τ} {( \frac{{R}_{0}}{l} )}^{6} ( 1 - x ) ≡ a ( 1 - x ) \qquad ( 9 )$$

where

$$a = 6 {( \frac{{R}_{0}}{l} )}^{6} ,$$

R₀ is the F\dot{or}ster Radius and l is the intermolecular spacing. For a hexagonal lattice

$$l = \sqrt{\frac{2}{\sqrt{3 } ρ}} \qquad ( 10 )$$

where ρ is the surface density. Because I_{F}(x)∝ [symbol] P_{F} [symbol] , I_{F}(1)≡I_{F} and

$$〈 {\sum }_{i} {k}_{i} 〉 = 0$$

when there are no quenchers then

$${I}_{F} ( x ) = \frac{{I}_{F}}{1 + τ 〈 {\sum }_{i} {k}_{i} 〉} \qquad ( 11 )$$

Making all substitutions into the original equation yields

$$I ( x ) = \frac{x {I}_{F}}{1 + a ( 1 - x )} + ( 1 - x ) {I}_{FQ} \qquad ( 12 )$$

The next task is to test the fitness of this model with the signal measured from the simulated surfaces. As discussed previously, the normalized fluorescence intensities generated a value for I_{FQ}=1.0 and I_{F}=6.23. The model is then fitted to the data with a single adjustable parameter ‘a’. With this set of data, ‘a’ was found to be 2.18 (FIG. 5). The last remaining question is whether or not this is a reasonable value. Under these experimental conditions, the surface probe density, ρ, is estimated to be about 5×10¹² molecules/cm², providing a value for l of 50 Å. Using this value of l together with a=2.18 yields a value for R₀ of 42 Å for the system described herein. This value falls nicely within the typical range of F\dot{or}ster radii (10-100 Å),^{33, 34} indicating that the 2.18 value for ‘a’ is reasonable.

The above Example clearly demonstrate that a structure-specific invasive cleavage reaction can be performed on planar substrates with single nucleotide specificity. Therefore, the present invention permits SNP genotypes to be identified unambiguously using a surface array format and FRET detection. Future work will focus on increasing the detection sensitivity of the surface invasive cleavage reaction.

### REFERENCES

(1) Stephens, J. C.; Schneider, J. A.; Tanguay, D. A.; Choi, J.; Acharya, T.; Stanley, S. E.; Jiang, R.; Messer, C. J.; Chew, A.; Han J. H.; Duan, J.; Carr, J. L.; Lee, M. S.; Koshy, B.; Kumar, A. M.; Zhang, G.; Newell, W. R.; Windemuth, A.; Xu, C.; Kalbfleisch, T. S.; Shaner, S. L.; Arnold, K.; Schulz, V.; Drysdale, C. M.; Nandabalan, K.; Judson, R. S.; Ruano, G.; Vovis, G. F. Science 2001, 293, 489-493.
(2) Wang, D. G.; Fan, J. B.; Siao C. J.; Bemo, A.; Young, P.; Sapolsky, R.; Ghandour, G.; Perkins, N.; Winchester, E.; Spencer, J.; Kruglyak, L.; Stein, L.; Hsie, L.; Topaloglou, T.; Hubbell, E.; Robinson, E.; Mittmann, M.; Morris, M. S.; Shen, N.; Kilburn, D.; Rioux, J.; Nusbaum, C.; Rozen, S.; Hudson, T. J.; Lipshutz, R.; Chee, M.; Lander, E. S. Science 1998, 280, 1077-1082.
(3) Brooks, A. J. Gene 1999, 234, 177-186.
(4) Kruglyak, L. Nat. Genet. 1999, 22, 139-144.
(5) Landegren U.; Nilsson, M.; Kwok, P. Y. Genome Res. 1998, 8, 769-776.
(6) Cyranoski D. Nature 2001, 410, 1013.
(7) Lyamichev, V.; Mast, A. L.; Hall J. G.; Prudent, J. R.; Kaiser, M. W.; Takova, T.; Kwiatkowski, R. W.; Sander, T. J.; Arruda, M. D.; Arco, D. A.; Neri, B. P.; Brow, M. A. D. Nat. Biotechol. 1999, 17, 292-296.
(8) Hall, J. G.; Eis, P. S.; Law, S. M.; Reynaldo, L. P.; Prudent, J. R.; Marshall, D. J.; Allawi, H. T.; Mast, A. L.; Dahlberg, J. E.; Kwiatkowski, R. W.; Arruda, M. D.; Neri, B. P. Proc. Natl. Acad. Sci. 2000, 97, 8272-8277.
(9) Lyamichev, V; Brow, M. A. D.; Dahlberg, J. E. Science 1993, 260, 778-783.
(10) Lyamichev, V. I.; Kaiser, M. W.; Lyamicheva, N. E.; Vologodskii, A. V.; Hall, J. G.; Ma, W. P.; Allawi, H. T.; Neri, B. P. Biochemistry 2000, 39, 9523-9532.
(11) Erlich, H. A.; Gelfand, D.; Sninsky, J. J. Science 1991, 252, 1643-1651.
(12) Weissensteiner, T.; Lanchbury, J. S. BioTechniques 1996, 21, 1102-1108.
(13) Erlich, G. D. PCR-based Diagnostics in Infectious Disease; Blackwell Scientific Publications: Oxford, England, 1994; pp 3.
(14) Heid, C. A.; Stevens, J.; Livak, K. J.; Williams, P. M. Genome Res. 1996, 6, 986-994.
(15) Kwiatkowski, R. W.; Lyamichev, V.; de Arruda, M.; Neri, B. Mol. Diagn. 1999, 4, 353-364.
(16) PRNewswire, Jan. 23, 2002. www.prnewswire.com.
(17) Griffin, T. J.; Hall, J. G.; Prudent, J. R.; Smith, L. M. Proc. Natl. Acad. Sci. 1999 96, 6301-6306.
(18) Stevens, P. W.; Hall, J. G.; Lyamichev, V.; Neri, B. P.; Lu, M.; Wang, L.; Smith, L. M.; Kelso, D. M. Nucl. Acids. Res. Methods Online 2001, 29, 77e.
(19) Tyagi, A.; Kramer, F. R. Nat. Biotechnol. 1996, 14, 303-308.
(20) Nickerson, D. A.; Taylor, S. L.; Fullerton, S. M.; Weiss, K. M.; Clark, A. G.; Stengard, J. H.; Salomaa, V.; Boerwinkle, E.; Sing, C. F. Genome Res. 2000, 10, 1532-1545.
(21) Guo, Z.; Guilfoyle, R. A.; Thiel, A. J.; Wang, R.; Smith, L. M. Nucl. Acids. Res. 1994, 22, 5456-5465.
(22) Glen Research Corporation, User Guide to DNA Modification and Labelling. 2001. Sterling, Va. www.glenres.com.
(23) Frutos, A. G.; Liu, Q.; Thiel, A. J.; Sanner, A. W.; Condon, A. E.; Smith, L. M; Corn, R. M. Nucleic Acids Res. 1997, 25, 4748-4757.
(24) Jordan C. E.; Frutos, A. G.; Thiel, A. J.; Corn, R. M. Anal. Chem. 1997, 69, 4939-4947.
(25) Brockman, J. M.; Frutos, A. G.; Corn, R. M. J. Am. Chem. Soc. 1999, 121, 8044-8051.
(26) Allawi, H. T.; SantaLucia J. Biochemistry 1997, 36, 10581-10594.
(27) SantaLucia, J. Jr.; Allawi H. T.; Seneviratne, P. A. Biochemistry 1996, 35, 3555-3562.
(28) Integrated DNA Technologies, Oligo Analyzer 2.5. 2001.
(29) Vainrub, A.; Pettitt, B. M. Chem. Phys. Letters 2000, 323, 160-166.
(30) Tyagi, S.; Bratu, D. P.; Kramer, F. R. Nat. Biotechnol. 1998, 16, 49-53.
(31) Forster, T. Discussions Faraday Soc. 1959, 27, 7-17.
(32) Stryer, L.; Haugland, R. P. Proc. Natl. Acad. Sci. U.S.A. 1967, 58, 719-726.
(33) Widengren, J. Schweinberger, E.; Berger, S.; Seidel, C. A. M. J. Phys. Chem. A, 2001, 105, 6851-6866.
(34) Didenko, V. V. Biotechniques 2001, 31, 1106-1121.
(35) Gray I C, Campbell D A, Spurr N K. 2000. Single nucleotide polymorphisms as tools in human genetics. Hum Mol Genet 9: 2403-8.
(36) IDT, 2000. Oligo Analyzer 2.5 (www.idtdna.com). Integrated DNA Technologies, Coralville, Iowa.
(37) Kaiser M W, Lyamicheva N, Ma W, Miller C, Neri B, Fors L, Lyamichev V I. 1999. A comparison of eubacterial and archaeal structure-specific 5′-exonucleases. J Biol Chem 274: 21387-94.
(38) Wilkins Stevens, P, Hall J G, Lyamichev V, Neri B P, Lu M, Wang L, Smith LM, Kelso D M. 2001. Analysis of single nucleotide polymorphisms with solid phase invasive cleavage reactions. Nucleic Acids Res 29: e77.

1. A method of detecting a single nucleotide polymorphism in a population of target nucleic acid molecules, the method comprising:
(a) providing:
(i) a cleavage agent;
(ii) a source of target nucleic acid molecules, at least one molecule of which comprises a first region and a second region, the second region being downstream of the first region, and further comprising a polymorphic nucleotide disposed between the first region and the second region, and wherein the first region, the polymorphic nucleotide, and the second region are contiguous;
(iii) a probe oligonucleotide comprising a 5′-terminal nucleotide and a 3′-terminus, wherein the probe oligonucleotide is immobilized at or near its 3′ terminus to an inert substrate, and wherein the probe oligonucleotide is complementary to the first portion and the polymorphic nucleotide of the target nucleic acid, with the 5′-terminal nucleotide of the probe oligonucleotide corresponding to and complementary to the polymorphic nucleotide of the target nucleic acid;
(iv) an upstream oligonucleotide comprising a 3′ terminal nucleotide and a contiguous 5′ portion, wherein the 5′ portion is complementary to the second portion of the target nucleic, and the 3′ terminal nucleotide corresponds to the polymorphic nucleotide in the target nucleic acid, and is or is not complementary thereto; and then
(b) contacting the cleavage agent, the target nucleic acid, and the upstream oligonucleotide to the immobilized probe oligonucleotide to create a reaction mixture under reaction conditions such that the probe oligonucleotide is annealed to the first region and the polymorphic nucleotide of the target nucleic acid and wherein at least the fraction of the 5′ portion of the upstream oligonucleotide is annealed to the second region of the target nucleic acid at a point contiguous to the polymorphic nucleotide in the target nucleic acid so as to create a cleavage structure, and wherein cleavage of the cleavage structure occurs to generate non-target cleavage products immobilized on the inert support; and
c) detecting cleavage of the cleavage structure, whereby the polymorphic nucleotide in the target nucleic acid is detected.

2. The method of claim 1, wherein in step (c), detecting cleavage of the cleavage structure comprises detecting the non-target cleavage products immobilized on the inert support.

3. The method of claim 1, wherein in step (c), detecting cleavage of the cleavage structure comprises a means for detection selected from the group consisting of means for detecting fluorescence, means for detecting mass; means for detecting fluorescence energy resonance transfer, means for detecting radioactivity, means for detecting luminescence, means for detecting phosphorescence, means for detecting fluorescence polarization, and means for detecting charge.

4. The method of claim 1, wherein in step (a)(iv) the 3′ terminal nucleotide of the upstream oligonucleotide is not complementary to the polymorphic nucleotide in the target nucleic acid.

5. The method of claim 1, wherein in step (a)(iv) the 3′ terminal nucleotide of the upstream oligonucleotide is complementary to the polymorphic nucleotide in the target nucleic acid.

6. The method of claim 1, wherein in step (a)(i) said cleavage agent comprises a structure-specific nuclease.

7. The method of claim 6, wherein the structure-specific nuclease comprises a thermostable structure-specific nuclease.

8. The method of claim 6, wherein the cleavage agent comprises a 5′-nuclease.

9. The method of claim 8, wherein the 5′-nuclease comprises a thermostable 5′-nuclease.

10. The method of claim 9, wherein a portion of the thermostable nuclease has an amino acid sequence that is homologous to a portion of an amino acid sequence of a thermostable DNA polymerase derived from a thermophilic organism.

11. The method of claim 10, wherein the amino acid sequence of the thermostable nuclease is homologous to a portion of an amino acid sequence of a thermostable DNA polymerase derived from a thermophilic organism selected from the group consisting of Thermus aquaticus, Thermus flavus, and Thermus thermophilus.

12. The method of claim 1, wherein the target nucleic acid comprises DNA.

13. The method of claim 1, wherein the target nucleic acid comprises RNA.

14. The method of claim 1, wherein the source of target nucleic acid comprises a sample containing genomic DNA.

15. The method of claim 14, wherein the sample is selected from the group comprising blood, saliva, cerebral spinal fluid, pleural fluid, milk, lymph, sputum, and semen.

16. The method of claim 1, wherein said reaction conditions comprise providing a source of divalent cations.

17. The method of claim 16, wherein sthe divalent cation is selected from the group consisting of Mn²⁺ and Mg²⁺ ions.

18. A method of detecting a single nucleotide polymorphism in a population of target nucleic acid molecules, the method comprising:
(a) providing:
(i) a cleavage agent;
(ii) a source of target nucleic acid molecules, at least one molecule of which comprises a first region and a second region, the second region being downstream of the first region, and further comprising a polymorphic nucleotide disposed between the first region and the second region, and wherein the first region, the polymorphic nucleotide, and the second region are contiguous;
(iii) a probe oligonucleotide comprising a 5′-terminal nucleotide and a 3′-terminus, wherein the probe oligonucleotide is immobilized at or near its 3′ terminus to an inert substrate, and wherein the probe oligonucleotide is complementary to the first portion and the polymorphic nucleotide of the target nucleic acid, with the 5′-terminal nucleotide of the probe oligonucleotide corresponding to and complementary to the polymorphic nucleotide of the target nucleic acid;
(iv) an upstream oligonucleotide comprising a 3′ terminal nucleotide, a contiguous 5′ portion, and a 5′ terminus, wherein the upstream oligonucleotide is immobilized at or near its 5′ terminus to the inert substrate at a point adjacent to the immobilized probe oligonucleotide, and wherein a fraction of the 5′ portion is complementary to the second portion of the target nucleic, and the 3′ terminal nucleotide corresponds to the polymorphic nucleotide in the target nucleic acid, and is or is not complementary thereto; and then
(b) contacting the cleavage agent and the target nucleic acid to the immobilized probe oligonucleotide and the immobilized upstream oligonucleotide to create a reaction mixture under reaction conditions such that the probe oligonucleotide is annealed to the first region and the polymorphic nucleotide of the target nucleic acid and wherein at least the 5′ portion of the upstream oligonucleotide is annealed to the second region of the target nucleic acid at a point contiguous to the polymorphic nucleotide in the target nucleic acid so as to create a cleavage structure, and wherein cleavage of the cleavage structure occurs to generate non-target cleavage products immobilized on the inert support; and
c) detecting cleavage of the cleavage structure, whereby the polymorphic nucleotide in the target nucleic acid is detected.

19. The method of claim 18, wherein in step (c), detecting cleavage of the cleavage structure comprises detecting the non-target cleavage products immobilized on the inert support.

20. The method of claim 18, wherein in step (c), detecting cleavage of the cleavage structure comprises a means for detection selected from the group consisting of means for detecting fluorescence, means for detecting mass; means for detecting fluorescence energy resonance transfer, means for detecting radioactivity, means for detecting luminescence, means for detecting phosphorescence, means for detecting fluorescence polarization, and means for detecting charge.

21. The method of claim 18, wherein in step (a)(iv) the 3′ terminal nucleotide of the upstream oligonucleotide is not complementary to the polymorphic nucleotide in the target nucleic acid.

22. The method of claim 18, wherein in step (a)(iv) the 3′ terminal nucleotide of the upstream oligonucleotide is complementary to the polymorphic nucleotide in the target nucleic acid.

23. The method of claim 18, wherein in step (a)(i) said cleavage agent comprises a structure-specific nuclease.

24. The method of claim 23, wherein the structure-specific nuclease comprises a thermostable structure-specific nuclease.

25. The method of claim 23, wherein the cleavage agent comprises a 5′-nuclease.

26. The method of claim 25, wherein the 5′-nuclease comprises a thermostable 5′-nuclease.

27. The method of claim 26, wherein a portion of the thermostable nuclease has an amino acid sequence that is homologous to a portion of an amino acid sequence of a thermostable DNA polymerase derived from a thermophilic organism.

28. The method of claim 27, wherein the amino acid sequence of the thermostable nuclease is homologous to a portion of an amino acid sequence of a thermostable DNA polymerase derived from a thermophilic organism selected from the group consisting of Thermus aquaticus, Thermus flavus, and Thermus thermophilus.

29. The method of claim 18, wherein the target nucleic acid comprises DNA.

30. The method of claim 18, wherein the target nucleic acid comprises RNA.

31. The method of claim 18, wherein the source of target nucleic acid comprises a sample containing genomic DNA.

32. The method of claim 31, wherein the sample is selected from the group comprising blood, saliva, cerebral spinal fluid, pleural fluid, milk, lymph, sputum, and semen.

33. The method of claim 18, wherein said reaction conditions comprise providing a source of divalent cations.

34. The method of claim 33, wherein the divalent cation is selected from the group consisting of Mn²⁺ and Mg²⁺ ions.

35. A method of detecting a single nucleotide polymorphism in a population of target nucleic acid molecules, the method comprising:
(a) providing:
(i) a cleavage agent;
(ii) a source of target nucleic acid molecules, at least one molecule of which comprises a first region and a second region, the second region being downstream of the first region, and further comprising a polymorphic nucleotide disposed between the first region and the second region, and wherein the first region, the polymorphic nucleotide, and the second region are contiguous;
(iii) a probe oligonucleotide comprising a 5′-terminal nucleotide and a 3′-terminal nucleotide, wherein the probe oligonucleotide is immobilized at or near one of the 5′- or 3′-terminal nucleotides to an inert substrate, and wherein the probe oligonucleotide is complementary to the first portion and the polymorphic nucleotide of the target nucleic acid, with the terminal nucleotide of the probe oligonucleotide not bound to the substrate corresponding to and complementary to the polymorphic nucleotide of the target nucleic acid;
(iv) an upstream oligonucleotide comprising a 3′ terminal nucleotide, a 5′ terminal nucleotide, and 3′ portion and a 5′ portion; wherein one of the 3′- or 5′ portions is complementary to the second portion of the target nucleic, and one of the 3′- or 5′-terminal nucleotides corresponds to the polymorphic nucleotide in the target nucleic acid, and is or is not complementary thereto; and then
(b) contacting the cleavage agent, the target nucleic acid, and the upstream oligonucleotide to the immobilized probe oligonucleotide to create a reaction mixture under reaction conditions such that the probe oligonucleotide is annealed to the first region and the polymorphic nucleotide of the target nucleic acid and wherein at least the fraction of the 3′ or 5′ portion of the upstream oligonucleotide is annealed to the second region of the target nucleic acid at a point contiguous to the polymorphic nucleotide in the target nucleic acid so as to create a cleavage structure, and wherein cleavage of the cleavage structure occurs to generate non-target cleavage products immobilized on the inert support; and
(c) detecting cleavage of the cleavage structure, whereby the polymorphic nucleotide in the target nucleic acid is detected.

36. A method of detecting a single nucleotide polymorphism in a population of target nucleic acid molecules, the method comprising:
(a) providing:
(i) a cleavage agent;
(ii) a source of target nucleic acid molecules, at least one molecule of which comprises a first region and a second region, the second region being downstream of the first region, and further comprising a polymorphic nucleotide disposed between the first region and the second region, and wherein the first region, the polymorphic nucleotide, and the second region are contiguous;
(iii) a probe oligonucleotide comprising a 5′-terminal nucleotide and a 3′-terminal nucleotide, wherein the probe oligonucleotide is immobilized at or near one of the 5′- or 3′-terminal nucleotides to an inert substrate, and wherein the probe oligonucleotide is complementary to the first portion and the polymorphic nucleotide of the target nucleic acid, with the terminal nucleotide of the probe oligonucleotide not bound to the substrate corresponding to and complementary to the polymorphic nucleotide of the target nucleic acid;
(iv) an upstream oligonucleotide comprising a 3′ terminal nucleotide, a 5′ terminal nucleotide, and 3′ portion and a 5′ portion, wherein the upstream oligonucleotide is immobilized at or near one of the 5′- or 3′-terminal nucleotides to the inert substrate; wherein one of the 3′- or 5′ portions is complementary to the second portion of the target nucleic, and one of the 3′- or 5′-terminal nucleotides corresponds to the polymorphic nucleotide in the target nucleic acid, and is or is not complementary thereto; and then
(b) contacting the cleavage agent and the target nucleic acid to the immobilized upstream oligonucleotide and the immobilized probe oligonucleotide to create a reaction mixture under reaction conditions such that the probe oligonucleotide is annealed to the first region and the polymorphic nucleotide of the target nucleic acid and wherein at least the fraction of the 3′ or 5′ portion of the upstream oligonucleotide is annealed to the second region of the target nucleic acid at a point contiguous to the polymorphic nucleotide in the target nucleic acid so as to create a cleavage structure, and wherein cleavage of the cleavage structure occurs to generate non-target cleavage products immobilized on the inert support; and
(c) detecting cleavage of the cleavage structure, whereby the polymorphic nucleotide in the target nucleic acid is detected.

37. A composition of matter comprising:
a metal substrate;
a probe oligonucleotide immobilized on the substrate at or near its 5′-terminus;
an upstream oligonucleotide immobilized on the substrate at or near its 5′-terminus;
wherein the probe oligonucleotide and the upstream oligonucleotide are immobilized on the substrates at points sufficiently close to one another to allow the probe oligonucleotide and the upstream oligonucleotide to participate cooperatively in an invasive cleavage reaction when the substrate is contacted with a cleavage agent and a target nucleic acid.

38. A composition of matter comprising:
a metal substrate;
a probe oligonucleotide immobilized on the substrate at or near its 3′-terminus;
an upstream oligonucleotide immobilized on the substrate at or near its 3′-terminus;
wherein the probe oligonucleotide and the upstream oligonucleotide are immobilized on the substrates at points sufficiently close to one another to allow the probe oligonucleotide and the upstream oligonucleotide to participate cooperatively in an invasive cleavage reaction when the substrate is contacted with a cleavage agent and a target nucleic acid.

39. A composition of matter comprising:
a metal substrate;
a probe oligonucleotide immobilized on the substrate at or near a terminus of the probe oligonucleotide;
an upstream oligonucleotide immobilized on the substrate at or near a terminus of the upstream oligonucleotide; and
wherein the probe oligonucleotide and the upstream oligonucleotide are immobilized on the substrates at points sufficiently close to one another to allow the probe oligonucleotide and the upstream oligonucleotide to participate cooperatively in an invasive cleavage reaction when the substrate is contacted with a cleavage agent and a target nucleic acid.
